(19) United States
(12) Patent Application Publication
(10) Pub. No.: US 2004/0028680 A1
(43) Pub. Date: Feb. 12, 2004

(54) Pharmacologic, therapeutic and diagnostic regulation of FGF-1 export

(75) Inventors: Thomas Maciag, Rockville, MD (US); Wilson H. Burgess, Clifton, VA (US); Francesca Tarantini, Olney, MD (US); Susan Gamble, Olney, MD (US); Anthony Q. Jackson, Landover, MD (US); Brian Hampton, Germantown, MD (US)

Correspondence Address: MORGAN, LEWIS & BOCKIUS LLP, 1701 MARKET STREET, PHILADELPHIA, PA, 19103-2921, US

(73) Assignee: Maine Medical Research Center

(21) Appl. No.: 10/353,458

(22) Filed: Jan. 29, 2003

Related U.S. Application Data
(62) Division of application No. 08/848,911, filed on May 1, 1997, now abandoned.
Application No. 08/848,911 is a continuation-in-part of application No. 08/640,711, filed on May 1, 1996, now abandoned.

(51) Int. Cl. A61K 39/395

(52) U.S. Cl. 424/145.1

(57) ABSTRACT

This invention relates to the regulation of FGF-1 export. In addition, this invention provides methods of regulating the export of FGF-1 from the cell membrane, which methods include regulating the Syn-1 molecule to affect the formation of the FGF-1:Syn-1 complex, regulating the enzyme or protease which cleaves Syn-1 in the FGF-1:Syn-1 complex to effect the release of FGF-1 from the cell membrane, or regulating the FGF-1:Syn-1 complex itself at or near the cell membrane. The invention also provides compositions related to the regulation of FGF-1 export.

### RELATED APPLICATIONS

This patent application is a continuation-in-part of U.S. application Ser. No. 08/640,711, filed May 1, 1996, the entire contents of which are herein incorporated by reference.

### STATEMENT OF GOVERNMENT RIGHTS IN THE INVENTION

Part of the work performed during the development of this invention utilized U.S. Government funds under NIH grants HL44336 and HL32348. The U.S. Government may have certain rights in this invention.

### FIELD OF THE INVENTION

The present invention relates to the regulation of FGF-1 export.

### BACKGROUND OF THE INVENTION

Members of the fibroblast growth factor (FGF) family are potent regulators of developmental, physiological and pathophysiologic events in mammals. Therefore, it is important to understand the mechanism by which these factors regulate diverse biological processes such as mesoderm formation, atherogenesis, angiogenesis and neurogenesis.

The FGF family has been the subject of numerous comprehensive reviews. (See, e.g., Burgess and Maciag, Annu. Rev. Biochem. 58: 575-606 (1989); Friesel and Maciag, FASEB J. 9: 919-925 (1995); Rifkin and Moscatelli, J. Cell Biol. 109: 1-6 (1989); Klagsbrun and Baird, Cell 67: 229-231 (1991).) The best characterized members of the FGF family are FGF-1 (acidic FGF) and FGF-2 (basic FGF). These two proteins are unusual growth factors in that they lack a classical signal sequence to direct their secretion through the conventional endoplasmic reticulum (ER)-Golgi apparatus. Ligation of a signal sequence to the FGF prototypes yields functional extracellular ligands with potent transforming potential in vitro and the ability to induce exaggerated angiogenic and neurotropic phenomena in vivo including the generation of atheroma-like lesions.

Because the mitogenic activities of FGF-1 and FGF-2 are mediated by a high affinity receptor at the plasma membrane surface, it has been proposed that a tightly regulated, yet unconventional, export pathway exists to regulate the export of these growth factors. Consequently, the regulation of human endothelial cell growth and the role of FGF-1 in mediating this process in vivo has been the subject of a number of studies. See, e.g., Maciag et al., Proc. Natl. Acad. Sci. USA 76: 5674-5678 (1979); Maciag et al., J. Cell Biol. 91: 420-426 (1981); Maciag et al., J. Cell Biol. 94: 511-520 (1982); Maciag et al., Science 225: 932-935 (1984).

The addition of a signal sequence to either FGF-1 or FGF-2 established the function of these FGF proteins as transforming genes in a variety of target cells. See, e.g., Rogelj et al. Nature 331: 173-175 (1988); Blam et al., Oncogene. 3: 129-136 (1988); Jouanneau et al., Proc. Natl. Acad. Sci. USA 88:2893-2897 (1991); Talarico and Basilico, Mol. Cell. Biol. 11:1138-1145 (1991); Forough et al., J. Biol. Chem. 268: 2960-2968 (1993). In vivo expression studies in the porcine iliac artery using a liposome:vector gene transfer method demonstrate an exaggerated hypertrophic response to the presence of extracellular FGF-1 including the formation of a prominent neointima containing numerous capillary and aorta-like structures (Nabel et al., Nature 362: 844-846 (1993)). In a similar manner, a transgene expressing FGF-4₍ₛₛ₎:FGF-1 chimera from the α-crystallin promoter active during lens development in the eye demonstrates a hypertrophic response including the population of the lens with blood vessels and nerve bundles (Overbeek et al., Development (1994)).

In both studies, the transfer of the wild-type FGF-1 cDNA failed to yield pathologic consequences. Prior studies (e.g., Thompson et al., Science 241: 1349-1352 (1988)) have suggested that FGF-1 may have evolved without a functional signal sequence because expression would result in inappropriate export during certain situations, resulting in the formation of exaggerated vascular and neuronal structures. Thus, it appears that the pathway for FGF-1 export is tightly regulated in order to control the angiogenic and neurogenic activities of the protein.

In view of the detrimental effects that may ensue as a result of FGF-1 export, effective regulation is needed. The present invention exploits the discovery that FGF-1 is exported in response to temperature stress as a FGF-1 homodimer:synaptotagmin (Syn)-1 complex, or as a cofactor-mediated FGF-1:Syn-1 complex, and the existence of a Syn-1 cleavage enzyme or protease (Syn-1-CE) to regulate export and further provides other related advantages.

### SUMMARY OF THE INVENTION

The present invention generally provides methods of regulating FGF-1 export. In addition, the invention provides methods of regulating the formation of an FGF-1:Syn-1 complex or a complex comprising FGF-1:Syn-1, comprising inhibiting, blocking or binding FGF-1:Syn-1 or a complex comprising FGF-1:Syn-1, thereby regulating the export of FGF-1. It also provides methods comprising stimulating or enhancing the formation of an FGF-1:Syn-1 complex or a complex comprising FGF-1:Syn-1, thereby regulating the export of FGF-1. Therapeutic agents useful in accordance with the aforementioned methods are also disclosed.

Therefore, in one embodiment, the present invention discloses a method of regulating FGF-1 export from a cell comprising administering a therapeutic agent in an amount sufficient to inhibit the export of FGF-1, wherein said therapeutic agent inhibits the formation of a complex comprising FGF-1, and wherein the formation of said complex is a prerequisite to FGF-1 export. In one variation of the foregoing embodiment, the complex further comprises Syn-1.

The present invention also discloses a method of regulating FGF-1 export wherein the therapeutic agent inhibits the release of FGF-1 from a complex comprising FGF-1 and Syn-1. In another variation, the therapeutic agent inhibits the release of FGF-1 from, or the association of FGF-1 with, an intracellular vesicle.

In another embodiment, the invention discloses a method of regulating FGF-1 export from a cell comprising administering a therapeutic agent in an amount sufficient to promote the export of FGF-1, wherein the therapeutic agent promotes the formation of a complex comprising FGF-1, and wherein the formation of the complex is a prerequisite for FGF-1 export. In one alternative embodiment, the complex further comprises Syn-1.

The present invention also discloses a method of regulating FGF-1 export wherein the therapeutic agent promotes the release of FGF-1 from a Syn-1 at or near the plasma membrane. In another variation, the therapeutic agent promotes the release of a complex comprising FGF-1 from an intracellular vesicle. In yet another embodiment, the therapeutic agent promotes the association of a complex comprising FGF-1 with an intracellular vesicle.

The invention further discloses a method of regulating FGF-1 export, comprising inhibiting a protease that cleaves Syn-1 in an FGF-1:Syn-1 complex, thereby inhibiting the export of the FGF-1:Syn-1 complex. In an alternative embodiment, a method of regulating FGF-1 export, comprising increasing the activity of a protease that cleaves Syn-1 in an FGF-1:Syn-1 complex, thereby promoting the release of FGF-1 at or near the plasma membrane, is disclosed.

In any of the various embodiments disclosed above, the therapeutic agent is selected from the group consisting of an antibody; an immunologically active fragment of an antibody; and an anti-inflammatory agent. In other variations, the therapeutic agent is a cofactor or an immunosuppressive agent.

In variations of the foregoing embodiments, the FGF-1-containing complexes may further comprise cyclophilin B or S100A13, or both. Therefore, in alternative embodiments, methods of regulating FGF-1 export wherein the therapeutic agent inhibits or enhances the interaction of cyclophilin B and/or S100A13 with an FGF-1-containing complex or with an intracellular vesicle, thereby inhibiting or promoting the release of FGF-1 at or near the plasma membrane.

The present invention also discloses a variety of therapeutic agents. Some of the disclosed agents stimulate increased export of FGF-1, while others inhibit the export of FGF-1. Therefore, one disclosed embodiment provides a therapeutic agent which regulates the export of FGF-1 from a cell, wherein the agent (a) inhibits export of FGF-1; (b) does not inhibit secretion of a leader sequence-containing protein; and C inhibits the binding between the FGF-1 and an intracellular vesicle.

In one embodiment, the invention discloses a therapeutic agent which inhibits the formation of an FGF-1:Syn-1 complex. In another embodiment, the disclosed therapeutic agent inhibits the release of FGF-1 from a complex comprising FGF-1. The invention also discloses therapeutic agents which inhibit the association of FGF-1 with an intracellular vesicle, or which inhibit the release of FGF-1 from an intracellular vesicle.

In any of the embodiments disclosed above, the therapeutic agent is selected from the group consisting of an antibody, an immunologically active fragment of an antibody, and an anti-inflammatory agent. In still other embodiments, the therapeutic agent is an immunosuppressive agent or a cofactor. Similarly, in any of the embodiments disclosed above, the therapeutic agent is a protein derived from a recombinant molecule.

The invention also discloses an isolated, purified protease that cleaves Syn-1 in an FGF1 :Syn-1 complex and which thereby regulates the export of FGF-1. In another embodiment, the present invention discloses an antibody capable of specifically binding to the protease or to the complex in such a fashion that it interferes with the action of the protease, thereby inhibiting the export of FGF-1.

Finally, the present invention discloses an FGF-1 homodimer:Syn-1 complex. In one variation, formation of the complex is cofactor-mediated.

### BRIEF DESCRIPTION OF THE FIGURES

FIG. 1 illustrates a working model of the FGF-1 export pathway as a diagrammatic flow chart of the events involved.

FIG. 2 is an immunoblot showing how a Syn-1 fragment is released in response to temperature stress. The immunoblot analysis shown used an antibody to Syn-1 on medium conditioned by heat shock (2 hr, 42° C.; lane 2) as compared with media that was not heat-shock conditioned (2 hr, 37° C.; lane 1) from FGF-1 NIH 3T3 transfectants, activated by addition of (NH₄)₂S0₄ and absorbed/eluted from heparin Sepharose. Recombinant p45 Syn-1 (100 ng) is shown in lane 3 (positive control); it has an apparent Mᵣ of about 42 kDa as shown in the immunoblot. In lane 2, a Syn-1 fragment—i.e., a p40 heparin-binding protein—having an apparent Mᵣ of about 40 kDa is shown.

FIG. 3 is a graph showing the binding of phosphatidylserine (pS) by recombinant p45 Syn-1. Recombinant human p45 Syn-1 was expressed in the pET3 expression system, purified by heparin affinity and RP-HPLC, iodinated using lactoperoxidase methods, and added to plastic wells coated with phospholipids as described in Perin et al., Nature 345: 260-3 (1990). Across the horizontal axis, the following abbreviations appear: Negative control, no phospholipid; pS, phosphotidylserine; pC, pl choline; pE, pL ethanol-amine; pI, pL inositol; and pG, pL glycerol. Results were reported as CPMs bound (± std deviation) after three washes with 0.1M Tris, pH 7.4, containing 0.15M NaCl; cpm's are shown on the vertical axis.

FIGS. 4A and 4B depict the release of FGF-1 as high Mᵣ complexes in response to temperature stress as shown by an immunoblot analysis of conditioned medium from heat shocked FGF-1 NIH 3T3 transfectants, activated by addition of either 0.1% DTT (FIG. 4A) or 90% (sat.) ammonium sulfate ((NH₄)₂S0₄) (FIG. 4B) adsorbed to heparin-SEPHAROSE, gradient eluted with NaCl. A non-reduced, limited SDS-PAGE FGF-1 immunoblot was performed using antiserum raised to FGF-1 on the samples as described by Jackson et al., PNAS 89: 10691-5 (1992). Mᵣs were assigned for comparative purposes and appear on the vertical axis. All samples shown are over heparin-SEPHAROSE.

FIGS. 5A and 5B show the results of native PAGE gel shift analysis of FGF-1 binding to phospholipids (pL). Native PAGE (pH 6.8) analysis was performed with varying amounts of phosphatidylserine (pS) in FIG. 5A and phosphatidylethanolamine (pEA) in FIG. 5B, but with a constant amount of [¹²⁵I]-FGF-1. Amounts of pS and pEA are expressed in nM increments across the top of each Figure. The pL were premixed with cold FGF-1, followed by addition of [¹²⁵I]-FGF-1 probe. The entire sample was subjected to electrophoresis, except the last lane in each gel. The last lane contained either pS or pEA containing cold FGF-1, loaded without probe; [¹²⁵I]-FGF-1 was added to the top of the lane immediately prior to electrophoresis.

FIGS. 6A and 6B are immunoblots showing Cu²⁺ induction of FGF-1 and Syn-1 heterodimers. In FIG. 6A, purified FGF-1 and p45 Syn-1 were incubated with and without Cu²⁺ and were then resolved by non-reduced SDS-PAGE. In FIG. 6B, procedures were identical to those used in 6A, except immunoblotting was performed with FGF-1 antibody. In FIGS. 6A and 6B, the results using FGF-1, FGF-1+p45 Syn, and p45 Syn (with and without Cu²⁺) are indicated across the horizontal axes; Mᵣ are shown on the vertical axes.

FIGS. 7A and 7B illustrate the resolution of FGF-1 and Syn-1 as a low-affinity heparin affinity high Mᵣ complex using non-reduced limited SDS-PAGE FGF-1/Syn-1 immunoblot analysis. The immunoblots shown in FIGS. 7A and B were prepared as described in FIG. 4. The immunoblot shown in FIG. 7A was probed with anti-FGF-1; the immunoblot shown in FIG. 7B was probed with anti-Syn-1. All samples shown are over heparin-SEPHAROSE. In both FIGS. 7A and 7B, NaCl concentration (M) is shown on the horizontal axes and Mᵣ on the vertical axes. Reference samples of FGF-1-alpha (FIG. 7A) and Syn (FIG. 7B) are also indicated.

FIG. 8 shows that antisense (γ) Syn-1 represses the release of FGF-1 in response to heat shock. Immunoblot analysis of conditioned medium from heat shocked FGF-1 NIH 3T3 transfectants cotransfected with antisense (γ) Syn-1 cDNA, as compared with control conditioned media; and cell lysates under comparable conditions. pXZ38 cells=control transfectants; #10 Syn-1 and #19 Syn-1 are low and high FGF-1 expressing γ-Syn-1, FGF-1 NIH 3T3 cell co-transfectants, respectively.

FIGS. 9A and 9B illustrate that antisense (γ)Syn-1 represses the growth of NIH 3T3 cells but does not attenuate the ability of exogenous FGF-1 to stimulate growth. FIG. 9A shows the results of a growth assay in which pXZ28 (FGF-1 transfected NIH 3T3 cells) are compared to pXZ38-Syn-1 antisense transfected cells. FIG. 9B shows results of a growth assay in which Syn-1 antisense transfected cells in response to FGF-1. The solid (closed) bars represent the addition of FGF-1 in FIG. 9B. Graphs representing the fetal bovine serum (FBS) growth response curves of FGF-1 and γ-Syn-1, FGF-1 co-transfectants as a function of FBS (FIG. 9A), and the growth response of γ-Syn-1, FGF-1 co-transfectants (FIG. 9B) as a function of FBS in the presence and absence of FGF-1 are shown. In both FIGS. 9A and 9B, serum (%, v/v) is illustrated on the horizontal axes, while cells/well (×10⁻⁴ and 10⁻⁵, respectively) are plotted on the vertical axes.

FIGS. 10A and 10B show that organelle restriction of intracellular FGF-1 attenuates the FGF-1 export pathway which is able to recognize cytosolic FGF-1 as a high Mᵣ β-gal chimera. 3T3 cell transfectants expressing FGF-1 either as a COOH-terminal β-gal chimera or as an NH₂-terminal SV4OT NLS COOH-terminal β-gal chimera were examined for the cytosolic and nuclear presence of the reporter gene using immunofluorescence microscopy with either an anti-β-gal antibody or X-gal staining (FIG. 10A). Total cell lysate processed for β-gal immunoblot analysis is shown at FIG. 10B.

FIG. 11 shows the results of putative FGF-1: β-Gal chimera associated proteins resolved by β-gal immunoprecipitation of metabolically labeled FGF-1:β-Gal NIH 3T3 cell transfectants. Cells were radiolabeled with (³⁵S)-met/cys, heat shocked (at 42° C., for 2 hr.), conditioned media was collected, cells were lysed, immunoprecipitated with anti-β-gal antibody and resolved by SDS-PAGE. Arrows mark the co-precipitated bands of interest. Times, temperature conditions, and media vs. lysates are indicated on the horizontal axis, while Mᵣ are indicated on the vertical axis.

FIG. 12 shows FGF-1 mutants and chimeric constructs prepared by recombinant circle PCR (RC-PCR; see Friedman, et al., Biochem. Biophys. Res. Commun. 198: 1203-8 (1994)). Stable 3T3 cell transfectants for each of these constructs have been isolated, and the cytosolic level of the individual FGF-I₂₁₋₁₅₄ mutant/chimeric proteins are similar as measured by FGF-1 immunoblot analysis. The traffic of the individual constructs are shown; these data are derived from cell fractionation/immunoblot analysis, X-gal staining and β-gal immunohistochemical analysis as described in FIG. 10. Export (indicated as “secretion”) of the individual construct was measured as described in FIG. 10. NTS=Nuclear Translocation Signal.

FIGS. 13A and 13B show the results of an FGF-1/Syn-1 immunoprecipitation/Syn-1 immunoblot analysis of ovine brain samples resolved by low heparin-sepharose affinity and Sephadex G-100 gel exclusion chromatography. Western blots of samples were obtained from a Sephadex G-100 column. The gel in FIG. 13A shows FGF-1 immunoprecipitation followed by Syn-1 immunoblot analysis. The gel in FIG. 13B shows Syn-1 immunoprecipitation followed by Syn-1 immunoblot analysis.

FIG. 14 illustrates a compilation of many Syn-1 and FGF-1 immunoblot and RP-HPLC analyses of the FGF-1:Syn-1 complex. The top panel represents RP-HPLC resolution, following immunoblot analysis of a heparin affinity chromatography identified fraction of ovine brain extract containing both FGF-1 and Syn-1. The middle panel shows a single peak representing the RP-HPLC resolution of the stable, pure protein complex comprising FGF-1 and Syn-1. The bottom panel, showing at least six (6) significant protein peaks, represents RP-HPLC resolution of the FGF-1:Syn-1 complex following chaotropic denaturation with 8M guanidinium HCl. The multiple peaks shown in the lower panel represent proteins having retention times that are different from that of either the FGF-1 or Syn-1 components of the protein complex.

FIG. 15 illustrates a deletion analysis of p45 Syn-1. The domain structure for p65 Syn1 is shown, and the domains include: the intravesicular domain, transmembrane domain and the extravesicular domain (facing cytosol) comprising the multimerization (M) domain, Ca²⁺-pS-binding (pS) domain, clatharin-binding (C) domain and the neurexin (N) domain. Individual domain deletion (A) Syn-1 mutants are shown as β-gal chimeric structures.

### DETAILED DESCRIPTION OF THE INVENTION

I. Definitions

Prior to setting forth the invention, it may be helpful to an understanding thereof to set forth definitions of certain terms that will be used hereinafter.

Export. As used herein, “export” of a protein refers to a metabolically active process of transporting a translated cellular product to the extracellular spaces or to the cell membrane by a mechanism other than by a leader sequence.

Purified. As used herein, “purified” means that the desired purified protein or fusion protein produces a single band on a conventional assay using an appropriate label, e.g., the protein stain, Coomassie blue.

Stress. As used in the present context, the hallmark of “stress” in the cell is the “heat shock response,” but the term includes a broad variety of stressors which may induce the response; e.g., amino acid analogs, puromycin, ethanol, heavy metal ions, arsenicals, tissue explantation, infection, etc. The reference to “heat shock” or “temperature stress,” therefore, is meant to include any stress that results in the export of FGF-1 or the formation of the FGF-1:Syn1 complex.

II. Description of Various Preferred Embodiments

The present invention relates to the discovery that FGF-1 is exported in response to various stimuli, including stressors (such as temperature stress), as an FGF-1 homodimer:synaptotagmin (Syn)-1 complex. This FGF-1:Syn-1 complex may also be cofactor-mediated, as described further hereinbelow. The present invention further discloses that complexation of FGF-1 with Syn-1 occurs under “non-stress” conditions as well and that complexes including FGF-1 and Syn-1 are an important part of the FGF-1 export and regulatory processes.

Insight into physiologic processes that are involved in the regulation of FGF-1 export evolved from the characterization of FGF-1 as a potent promoter of angiogenesis in vivo (see, Folkman and Klagsbum, Science 235:442-447 (1987)). The present invention provides further insights and describes methods of inhibiting, blocking or binding either the FGF-1:Syn-1 complex before the FGF-1 is released from the cell membrane, or of inhibiting, blocking or binding the protease(s) that allow release of FGF-1 from its complex with Syn-1, thus decreasing export of FGF-1.

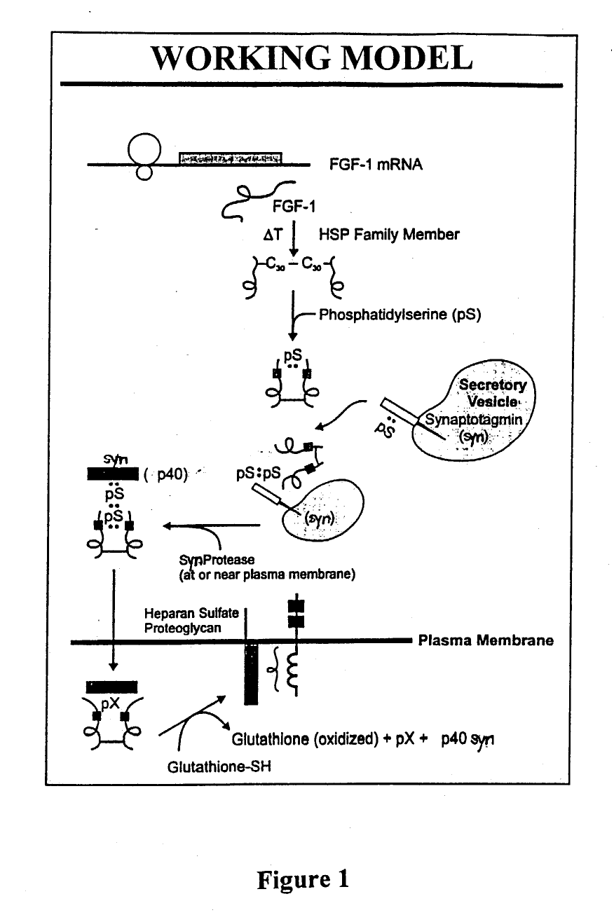

It should be appreciated that the present invention is not limited by the proposed models and mechanisms described herein. Thus, it should be understood that models such as the one shown in FIG. 1, for example, present a working model showing the involvement of FGF-1 in complex formation, the FGF-1:Syn-1 complex, the effect of a proteolytic cleavage of the complex at or near the cell membrane, and the like, all of which facilitates understanding of the invention.

The existence of an FGF-1:Syn-1 complex, the effect of proteolytic cleavage of the complex at or near the cell membrane, and the effect of release of the FGF-1 molecule from the cell membrane are all disclosed herein. Various working examples appear below and present data showing the following, for example: (I) the FGF-1 homodimer is associated with a p45 fragment of p65 Syn-1 in medium conditioned by heat shock; (ii) expression of antisense to Syn-1 completely attenuates the release of FGF-1 in response to temperature stress; (iii) mutagenesis of the FGF-1 cys residues demonstrate the functional importance of cys30 for the release of FGF-1 in response to temperature stress; (iv) the extracellular FGF-1 complex, resolved by the immunoprecipitation of an FGF-1: β-galactosidase (gal) chimera released into the conditioned medium in response to heat shock from NIH 3T3 cell transfectants, contains novel (³⁵S)-met-labeled polypeptides including a putative candidate for an extracellular form of (³⁵S)-Syn-1; and (v) the resolution of p45 Syn-1:FGF-1 homodimer as a low affinity heparin-binding complex from ovine brain. The invention further demonstrates that the FGF-1 export pathway is non-conventional and that it is possible to inhibit or enhance the export of FGF-1 at several points along the FGF-1 export pathway.

As illustrated in FIG. 1, the FGF-1 translation product, in response to temperature, unfolds and undergoes oxidation to form a homodimer specifically utilizing cyS₃₀. It is important to understand that FGF-1 is not a heat-shock response gene; rather, the Figure illustrates our finding that at 42° C., FGF-1 specifically associates with phosphatidylserine (pS) at the COOH-terminal, heparin-binding domain. (See, e.g., Burgess et al., J. Cell Biol. 111:2129-2138 (1990).) As disclosed hereinbelow (see, e.g., Example 10), the model we have presented in FIG. 1 is a useful model in other “stress” situations and in “non-stress” situations, as well.

The recent observation that FGF-1 at 42° C. undergoes a conformation change which enables it to attain “molten globule” character and associate with acidic phospholipids within this domain (see, Mach and Middaugh, Biochemistry 34:9913-9920 (1995)) is consistent with this premise and supports the proposed FGF-1 export pathway (FIG. 1).

The FGF-1 homodimer:pS complex then associates with either (I) Syn-1; (ii) the Syn1:pS and/or (I); and/or other proteins. However, it is presumed that FGF-1 utilizes the cytosolic face of the conventional exocytotic pathway. Release from this constraint may be carried out by a protease which solubilizes the FGF-1 homodimer:pS:Syn-1 complex as a cytosolic structure. As used herein, the term “protease” includes native or modified enzymes, proteolytic complexes, or functionally active fragments thereof, whether those fragments are in isolated form or in a complex, as long as said variants or fragments retain proteolytic activity.

Evidence from the platelet and red blood cell research suggests that a “flippase” complex is able to “flip” pS from the extracellular and intracellular membrane locales (see, Zachowshi, Biochem. J. 294:1-14 (1993)). However, regardless of the mechanism involved, the intracellular FGF-1 complex transverses the plasma membrane where it is released as a latent heparin- and FGFR-1-binding protein. Activation by a reducing agent is required for extracellular FGF-1 to associate with the heparin sulfate proteoglycan or other acidic cell-associated extracellular macromolecular structures, which in turn enables it to associate with its high affinity FGFR to induce receptor-mediated tyrosine phosphorylation.

A. FGF-1

1. Translational Efficiency of an Endogenous FGF-1 Transcript Requires the Use of Cells Transfected with Synthetic FGF-1 cDNA for FGF-1 Export.

As described above, FGF-1 is a member of a group of proteins that lack a canonical leader sequence and which are often identified as “leaderless proteins.” As used herein, “leaderless protein” refers to a protein or polypeptide that is found in an extracellular environment but lacks a canonical leader sequence. A leader sequence mediates translocation into the ER and is recognized by signal recognition proteins (SRP). Proteins in the extracellular environment include exported proteins found in extracellular spaces, as well as proteins that are membrane bound, but not as an integral membrane protein. The prototypic leader sequence has an amino-terminal positively charged region, a central hydrophobic region, and a more polar carboxy-terminal region (see, von Heijne, J. Membrane Biol. 115:195-201, 1990). Leaderless proteins include FGF-1, FGF-2 (and active fragments thereof), as well as F100, interleukin-1α, interleukin-1β, vas deferens protein, platelet-derived endothelial cell growth factor (PD-ECGF), ciliary neurotrophic factor (CNTF), thymosin, parathymosin, 14.5 kDa lectin (L14), transglutaminase, thioredoxin-like protein, sciatic nerve growth-promoting activity, factor XIIIa, mammary-derived growth inhibitor, galectin, rhodanase, HIV tat, and the like. Within the context of the present invention, leaderless proteins include naturally-occurring proteins as well as proteins that are engineered to lack a leader sequence, but are exported. The terms “signal sequence,” “leader peptide,” and “leader sequence” are used interchangeably herein.

In addition, consistent with usage in the art, FGF-1 is representative of molecules identified as ligand molecules. As used herein, the term “ligand” is meant to include any molecule or portion thereof capable of recognizing (having a binding affinity to) a particular binding molecule. Ligands which recognize a particular binding molecule naturally exist or can be prepared. Illustrative ligands include, but are not limited to, FGF-1 biotin, antigens, hormones, etc.

FGF-1 is useful in promoting the repair and healing of soft tissue injuries including burns, lacerations, and cutaneous ulcerations, as well as injuries to the cornea. The up-regulation of FGF-1 also promotes the healing of bone fractures, torn ligaments, torn or inflamed tendons, and inflammation of bursas. Similarly, stimulation of FGF-1 release facilitates the regeneration of cartilage and cartilaginous tissue.

FGF-1 also promotes central and peripheral nerve tissue repair and maintenance. Thus, it is not surprising that complexes containing FGF-1 and Syn-1 have now been identified in the brain, as discussed further in the Examples that follow. Enhanced FGF-1 release also has a beneficial or therapeutic effect in the amelioration of vascular injury, as FGF facilitates vascular tissue repair and the growth of new blood vessels (i.e., angiogenesis). Other beneficial effects of increased FGF-1 production are presented in U.S. Pat. Nos. 5,223,483 and 5,401,832, for example (the disclosures of which are incorporated by reference herein).

Therefore, in one embodiment of the present invention, a therapeutically effective amount of a therapeutic agent which increases FGF-1 export is an amount which stimulates FGF-1 export to the degree that therapeutic levels of FGF-1 effective to bring about the foregoing beneficial effects are reached.

2. FGF-1 Release in Response to Temperature Stress in Vitro.

FGF-1 is released by FGF-1 NIH 3T3 cell transfectants in response to heat shock, for example, as a non-heparin-binding and biologically inactive structure (Jackson et al., Proc. Natl. Acad. Sci. USA 89: 10691-10695 (1992)); the FGF-1 so produced is also referred to herein as latent FGF-1. In the foregoing example, “heat shock” conditions to whiuch the transfected cells were exposed consisted of a 2-hour exposure to a temperature of 42° C. In contrast, NIH 3T3 cells transfected with either FGF-2 or the IL-1 a precursor did not release these proteins in response to temperature stress, confirming that the release of FGF-1 is due to a unique mechanism and is not due to either an artifactual or passive process.

The heparin-binding and mitogenic activities of latent extracellular FGF-1 could be activated by fractionation of the heat-shocked conditioned medium with (NH₄)₂SO₄. While the precise mechanism by which (NH₄)₂SO₄ achieves the activation of latent extracellular FGF-1, it was observed that the structure of FGF-1 as predicted from crystallographic data demonstrates the presence of a sulfate-binding site (see Zhu et al., Science 251: 90-93 (1991)).

Further, the release of latent FGF-1 in response to temperature stress involves both transcription and translation processes since the release of latent FGF-1 could be inhibited by either actinomycin D or cycloheximide (Jackson et al., PNAS USA 89: 10691-10695 (1992)). However, neither the steady-state mRNA, nor the protein levels of either the endogenous or transfected FGF-1 transcripts were altered by heat shock in vitro, which is consistent with the lack of a heat shock response element within the FGF-1 gene or within the pMEXneo expression vector.

3. FGF-1 Homodimer Formed in Response to Copper Oxidation.

Cu²⁺ is a potent potentiator of angiogenesis in vivo (see, Gullino et al., Anticancer Res. 6:153-158 (1986)). Cu²⁺ oxidation induces FGF-1, but not FGF-2 or IL-Iα, to form a homodimer in vitro, which binds poorly to immobilized heparin (elution near 0.4M NaCl), and is not biologically active as a mitogen. However, it is possible to recover the heparin-binding and mitogenic activity from the Cu²⁺-induced FGF-1 homodimer by incubation of the FGF-1 homodimer with reducing agents, such as DTT.

The FGF-1 protein activated with DTT (I) is able to bind to immobilized heparin, (ii) is an active mitogen, and (iii) its activity is potentiated by heparin. In addition, the biological activity of latent FGF-1 released in response to temperature stress has been shown to be activated by 1 mM reduced glutathione, but not with 1 mM oxidized glutathione, suggesting that FGF-1 is released in response to heat shock as a latent homodimer. Indeed, FGF-1 homodimers have been purified from medium conditioned by heatshock.

Recombinant FGF-1_{CF} protein—an FGF-1 mutant with all cysteines altered—was found to be biologically active and bound immobilized heparin at a similar salt concentration as FGF-1 (Jackson et al. (1995)). The recombinant FGF-1_{CF} protein was expressed in bacteria from the pET3c vector containing FGF-1 cys-free (CF) mutant, in which the three cysteine (cys) residues in FGF-1 were converted to serine (ser) residues.

However, FGF-1_{CF} expressed in mammalian cells from the pMEXneo plasmid was not released under temperature stress conditions in which FGF-1 is released, despite the cytosolic levels of the FGF-1_{FC} mutant being approximately three-fold higher than the cytosolic levels of wild-type FGF-1. Thus, cysteine residues are required for release of latent FGF-1.

4. Agents Which Inhibit ER:Golgi Function Potentiate, rather than Inhibit the Release of Latent FGF-1 in Response to Heat Shock.

The pharmacologic agents brefeldin A, methylamine and verapamil are potent inhibitors of ER:Golgi function (Lippincott-Schwartz et al., Cell 56: 801-813 (1989)), exocytosis (Saraste et al., Proc. Natl. Acad. Sci. USA 83:6425-6229 (1986)), and the drug resistant release pathway (Endicott et al., Annu. Rev. Biochem. 58: 137-171 (1989)). However, when exposed to temperature stress, brefeldin A surprisingly potentiated, rather than repressed, the release of latent FGF-1 in response to heat shock (Jackson et al. (1995)). Similar data were obtained with methylamine- and verapamil-pretreated NIH 3T3 cell FGF-1 transfectants prior to heat shock (data not shown).

These results demonstrate that the temperature stress mechanism utilized by FGF-1 for secretion is non-conventional. Further, the mechanism of FGF-1 release appears to be unique to FGF-1, since it has been reported that the release of FGF-2 is repressed by brefeldin A, methylamine, and verapamil and that FGF-2 is not released in response to heat shock (Mignatti et al., J. Cell. Physiol. 151:81-93 (1992).

B. Synaptotagmin (Syn-1)

Our findings confirm that Syn-1 is involved in the FGF-1 export pathway. FGF-1 purification procedures utilizing bovine brain (see, Maciag et al., J. Biol. Chem. 257:5333-5336 (1982)) lead to very small amounts of FGF-1. Additionally, the extraction of FGF-1 from tissue using low ionic strength and neutral pH buffers yielded Sephadex G-100 gel exclusion profiles containing FGF-1 biological activity as a high Mᵣ fraction (Mᵣ>80,000), and as a low Mᵣ fraction (Mᵣ˜20,000). Collection of the high Mᵣ FGF-1 fraction followed by (NH₂)₄SO₄ fractionation and a second round of Sephadex G100 chromatography yielded exclusively a low Mᵣ fraction containing FGF-1 mitogenic activity. The transition from high Mᵣ to low Mᵣ using (NH₄)₂ SO₄ fractionation established that brain-derived, native FGF-1 exists as a large Mᵣ complex.

Using other methods, a heparin-binding protein (p45) which eluted with FGF-1 was obtained and resolved from FGF-1 by reversed phase HPLC (see, Burgess et al. (1985)). Although the DNA sequence was determined at the time by one of the present inventors, protein p45 remained functionally unidentified until shown recently to be an amino terminally truncated form of synaptotagmin (Syn)-1.

Syn-1 was initially identified as part of the mechanism utilized by synaptic vesicles to dock at nerve terminal endings (Perin et al., J. Biol. Chem. 266:623-629 (1991)). Its structure was deduced from a cDNA clone and predicts a protein (p65) with an NH₂-terminal domain (p45), which was anchored to Golgi vesicles by a transmembrane domain extending into the vesicle by a short COOH-terminal intervesicular domain. An additional structural feature predicted from the cDNA sequence was the apparent lack of a classical signal sequence to direct the transport of Syn-1 into the ER-Golgi apparatus. Furthermore, nine of the ten amino acid residues at the NH₂-terminus of Syn are identical to the NH₂-terminal sequence present in the yeast mating factor “α,” a protein which is released, but which also does not contain a classical signal sequence (Astell et al., Cell 27:15-23 (1981)).

Because the expression of Syn-1 appears requisite for the release of FGF-1 in response to heat shock and Syn-1 is associated with FGF-1 as a high Mᵣ complex in medium conditioned by temperature stress, experiments have been designed to confirm the structural and mechanistic basis for this interaction. Many Syn-1 sequences have already been identified, including for example, human, rodent and bovine Syn-1 sequences. These sequences are highly conserved (95%). From the sequence of p29 Syn-1, RT-PCR may be used to prepare a cDNA encoding p29 Syn-1 for expression as a GST fusion protein in E. coli using the pET3c expression vector as described above.

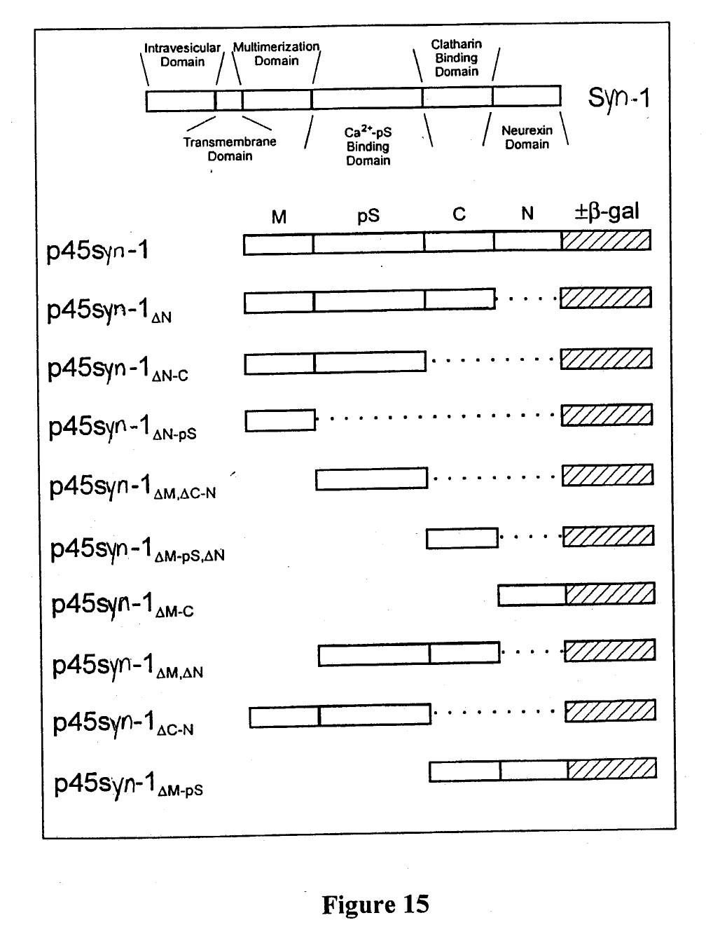

The Syn-1 protein can be divided into four domains (FIG. 15). Since β-Syn-1 expression inhibits FGF-1 export in response to heat shock, we expect that the “opposite” experiment will demonstrate that the exaggeration of Syn-1 expression in stable FGF-1 transfectants, like brefeldin A pretreatment, will result in an increase in the level of FGF-1 released into medium conditioned by heat shock. This permits confirmation of any result from the Syn-1 domain deletion effort by the expression of this domain in the same system. Thus, the FGF-1: β-gal ECV cell transfectants are co-transfected with either the complete sense Syn-1 ORF or the p45 Syn-1 ORF and stable co-transfectants obtained. When the temperature-induced FGF1 export pathway is augmented in these cells (as determined by FGF-1 immunoblot and ELISA), it confirms an increased level of the soluble p45 Syn-1 fragment in medium conditioned by temperature using Syn-1 immunoblot analysis. The domain deletion strategy is set forth in FIG. 15. The FGF-1:β-gal ECV cell transfectants are co-transfected with individual deletion mutants.

While it is expected that the expression of the “appropriate” deletion domain mutant will attenuate the release of FGF-1, the level of attenuation may not be as significant as previously described using the γ-Syn-1 expression plasmid. Therefore, two strategies have been designed to ascertain whether the FGF-1 export pathway is functioning near saturation, or if not, if there are insufficient intracellular levels of the individual domain deletion Syn-1 mutants to overcome the function of the endogenous Syn-1 protein. The within-described experiments (I) employ the use of a cell line wherein the expression of FGF-1 is reasonable as a system to stably express both the γ-Syn-1 (AUG start site-oriented γ-Syn-1 construct) and sense Syn-1 domain deletion mutant constructs; and (ii) allow one to obtain stable FGF-1:β-gal ECV transfectants which have been co-transfected with the appropriate Syn-1 domain lacking the remainder of the Syn-1 sequence.

The Syn-1 domain deletion strategy is supplemented by an independent point mutagenesis study in which the role of the Syn-1-CE cleavage site in the FGF-1 release pathway is assessed, establishing whether a Syn-1 point mutant ultimately functions as a dominant negative regulator of FGF-1 release in response to temperature stress.

C. Other Members of the FGF-1:Syn-1 Complex

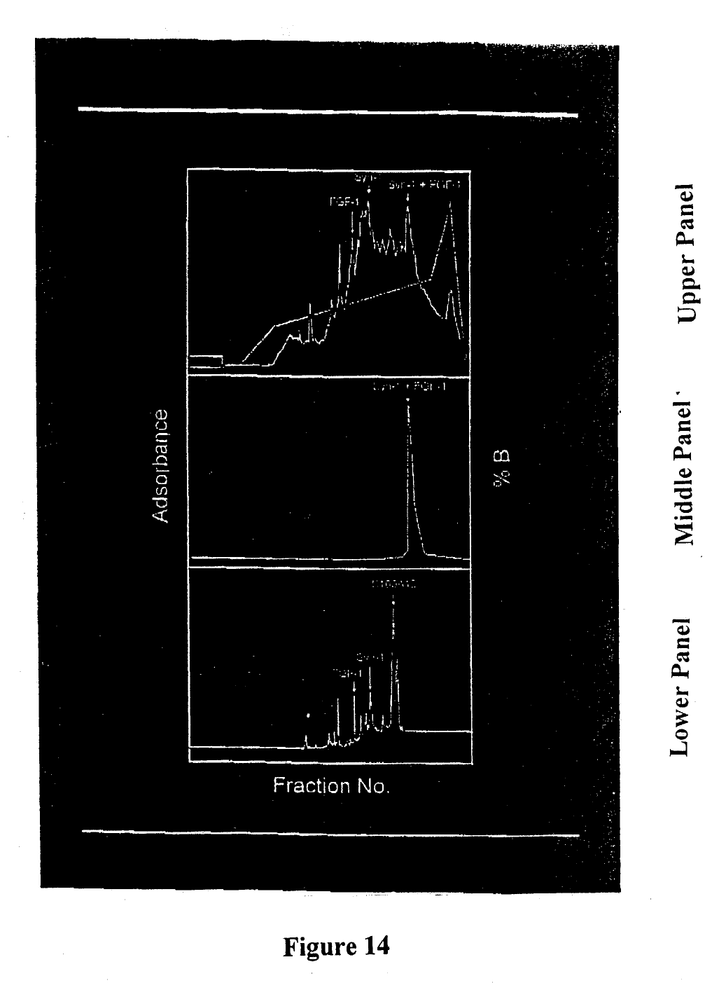

In addition to the FGF-1 and Syn-1 members of the stable FGF-1:Syn-1 complex, tissue/organ extracts prepared at neutral pH, when treated with denaturing agents generated multiple additional peaks representing distinct proteins having different elution retention times upon resolution by RP-HPLC. See, e.g., Example 9 and FIG. 14. Thus, treatment of a tissue preparation comprising the FGF-1:Syn-1 complex with a chaotropic agent (e.g., 8M guanidinium HCl) demonstrated that FGF-1 is not only complexed with Syn-1 in neutral pH extracts of ovine brain, but also with other proteins. Further, it was determined that this complex is remarkably stable to exposure to RP-HPLC solvents.

Conventional protein sequencing methods, however, failed to identify the other proteins associated with either FGF-1, Syn-1 or both in the Syn-1:FGF-1 complex (FIG. 14, bottom panel). Moreover, Edman degradation of the individual peaks resolved by RP-HPLC did not yield an NH₂-terminal sequence, showing that each of the components of the FGF-1:Syn-1 complex contain a blocked NH₂-terminus, including FGF-1 and Syn-1. This determination suggested that the purification of the multiprotein×FGF-1:Syn-1 complex can be achieved under conditions which do not result in the hydrolytic modificaton of the NH₂-terminus of any component of this complex.

Since the identity of the individual protein components of the FGF-1:Syn-1 complex could not be accessed by direct NH₂-terminal sequence analysis, peptide maps of those peaks exhibiting prominent absorbance profiles were obtained. Analysis of the most prominent peaks by NH₂-terminal sequence analysis of multiple proteinase K-derived peptides identified the first peak to be identical to the ovine homolog of human S100A13. Similar analysis of the second prominent absorbance peak demonstrated it to be identical to the ovine homolog of human cyclophilin B. Thus, the present data clearly identifies S100A13 and cyclophilin B to be components of the FGF-1:Syn-1 complex.

S100A 13 is a recently identified member of the human S100A gene family with well conserved structural features. Characterization of the isolated S100A 13 gene has shown that it contains the prerequisite structural motifs identifying the S100A gene family members as Ca²⁺-, calmodulin-, and acidic phospholipid-binding proteins. Interestingly, S100A proteins have been implicated as chemotactic and neurotrophic agents, which are activities also associated with FGF-1. Although it is known that like FGF-1, S100A proteins are able to modify intracellular tyrosine phosphorylation (MAPK-dependent) and transcriptional events, no known receptor for the family members has been identified. Further, S100A gene expression is diagnostic for metastatic human melanoma. While FGF-1 is well characterized as a mitogen for human melanocytes, its secretion has been implicated in the regulation of human metastatic tumor potential due to its ability to stimulate angiogenesis in vivo.

In addition, S100AI3 is a cysteine-free structure which is exported even though, like FGF1, it lacks a classical signal peptide sequence to direct it through the conventional ER-Golgi apparatus- mediated exocytotic pathway. Nevertheless, S100A proteins are well characterized as a calmodulin-binding protein, and are involved in the regulation of the filamentous cytoskeleton. Data suggests that FGF-1 may utilize the cytosolic side of conventional Syn-1 containing vesicles for traffic to the inner surface of the plasma membrane. Thus, S100A13 may serve as a conduit between the exocytotic organelle and the calmodulin-rich F-actin cytoskeleton. In addition, data suggest that (i) the biologic activities previously associated with the S100A gene family members may actually be the result of an FGF gene family contaminant found in relatively low specific activity preparations, and (ii) S100A antagonists could prove useful to limit the release of FGF-1.

The identification of cyclophilin B (CpB) as a component of the ovine brain-derived FGF1:Syn-1 complex is also interesting. CpB is a cyclosporin A1 (CSA)-binding protein, which was initially identified as a heat shock-induced peptidylproline CIS-trans isomerase (hsp40) involved in protein-folding and protein-protein interactions. CsA is well recognized as an immune suppressor in humans, having novel tissue/organ-specific side effects, including enhanced peripheral nerve repair in man. Interestingly, FGF-1 is not only well recognized as a cell survival factor, but it is also a potent neurotropic agent in vivo.

Different cyclophilins are located in different compartments of the cell. Cyclophilin B is known to reside in the ER, since it does contain a signal peptide sequence. However, it has been suggested that cyclophilin B is able to traffic through the ER-pre-Golgi pathway in association with secreted proteins inside the secretory vesicle. Therefore, cyclophilin B isolated from the brain as part of the FGF-1:Syn-1 complex may be associated with the intravesicular portion of Syn-1, and may be able to traffic with the vesicle-associated complex. Cyclophilin in this context may be necessary to regulate the assembly of the Syn-1 monomer into a dimeric or multimeric structure, to regulate and/or direct the traffic of the secretory vesicles under stress conditions, or to control the proteolytic formation of the p40 fragment from p65 Syn-1.

In summary, the present data provide evidence to support an association of Syn-1 and FGF-1 as a multiprotein complex in neural tissue in vivo, and to confirm the characterization of the FGF-1:Syn-1 complex derived from in vitro systems. In addition, because two additional proteins present within the FGF-1:Syn-1 complex have been identified as S100A13 and cyclophilin B, it is assumed that agents which agonize or antagonize this association will provide useful reagents for the inhibition and/or activation of the FGF-1 secretion pathway in vitro and in vivo.

D. Syn-1 Protease

In another embodiment of the invention, the protease responsible for cleaving and releasing Syn-1 into the conditioned medium of heat-shocked NIH 3T3 cells as a proteolytic fragment (p45) of the p65 Syn-1 precursor is disclosed. Although not intended to be limiting, preferred FGF-1 constructs for demonstrating the proteolytic effect are the FGF-1₁₋₁₅₄ and FGF1₁₋₁₅₄:β-gal EC and SMC transfectants. This enzyme is of particular interest because of its ability to cleave Syn-1 on the cytosolic side of the transmembrane sequence, which would effectively result in the formation of a soluble cytosol-derived FGF-1 secretory complex.

Once the active site character of the protease is known, conventional methods, using e.g., fluorescent esters and amides as alternative substrates for the protease, will permit the elucidation of its kinetic parameters, as well as permit the definition of an enzyme unit for the determination of specific activity. A combination of protease-inhibitor affinity chromatography, heparin-Sepharose chromatography, and reversed phase HPLC methods is capable of providing sufficient protein with high specific activity for microsequence analysis, which will provide direct access to the cDNA encoding the protease (through e.g., conventional cDNA cloning using the λgt 11 HUVEC/NIH 3T3 cell libraries as described (Zhan et al., J. Biol. Chem. 268:24427-24431 (1993); Zhan et al., J. Biol. Chem. 269:20221-20224 (1994)).

Assignment of the peptide bond in Syn-1 cleaved by the protease may be readily performed by sequence analysis of the p45 product of the Syn-1 (p65) precursor and the protease reaction mixture since the primary structure of all components are known.

Should the cell culture-based strategy for the purification of the Syn-1-CE prove to be cumbersome, an alternative method is available utilizing the bovine brain as a resource to purify and characterize the protease as reported by Maciag et al. (J. Biol. Chem. 257:5333-5336 (1982)).

Since it was possible at that time to collect the high Mᵣ FGF-1 fractions using gel exclusion chromatography, incubated at pH 4.0 to convert the high Mᵣ form of FGF-1 to its conventional low Mᵣ (20 kDa) form, and resolve the low Mᵣ form of FGF-1 by gel exclusion chromatography and isoelectric focusing, it is applicable to the FGF-1 purification strategy since the high Mᵣ form of FGF-1 may contain FGF-1 complexed to Syn-1 as an FGF-1 homodimer (see, e.g., Example 9).

A screen of the non-adsorbed and gradient-eluted heparin-Sepharose fractions will resolve the presence of a fraction(s) containing Syn-1 cleavage enzyme (Syn-1-CE). Further, use of the (¹²⁵I)-p65-Syn-1 to p45-Syn-1 conversion assays described above will not only define the post-heparin-Sepharose fraction(s) containing Syn-1-CE activity, but will also permit the use of neuronal tissue to purify sufficient Syn-1-CE protein for structural characterization by microsequencing and utilize a human brain stem cDNA library (previously used to clone the FGF-1 cDNA) to isolate and characterize the Syn-1-CE cDNA. Organ extracts have been shown to be a reliable and stable source of novel proteins (i.e., Syn-1, FGF-1, etc.).

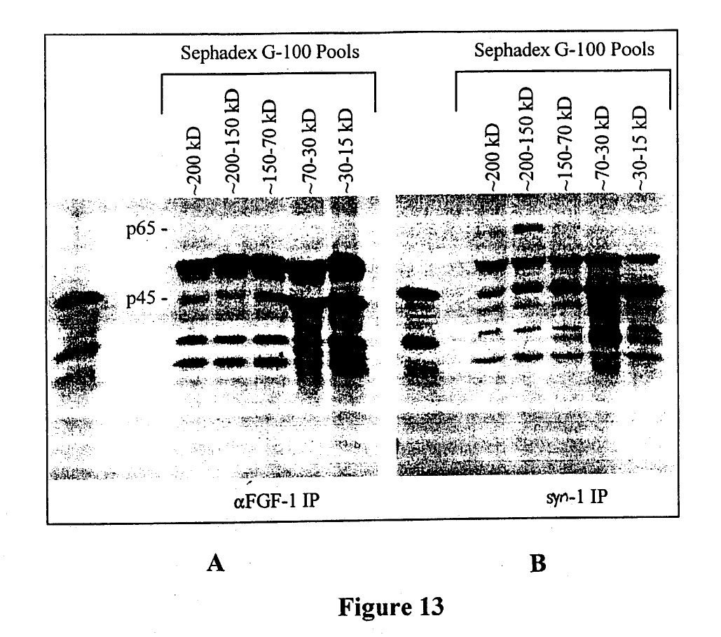

An FGF-1 antibody was able to resolve p45 Syn-1 and low Mᵣ Syn-1 fragments (see, FIG. 13), but not p65 Syn-1 by Syn-1 immunoblot analysis. In contrast, while p65 Syn-1 was readily resolved by Syn-1 immunoprecipitation followed by Syn-1 immunoblot analysis in the high Mᵣ post-Sephadex G-100 pools, p65 Syn-1 was not detected in the high M post-Sephadex G-100 pools by FGF-1 immunoprecipitation (FIG. 13). Thus, it is possible that the FGF-1 homodimer may not be able to associate with p65 Syn-1, but may associate with p45 Syn-1.

Moreover, since both FGF-1 and Syn-1 immunoprecipitates were able to resolve lower Mr Syn-1 fragments (p29 and p35) and the lowest Mᵣ Syn-1 fragment, p29, it is possible that the Syn-1 fragment may define the minimal Syn-1 sequence able to associate with the FGF-1 homodimer. To confirm this, p29 Syn-1 may be purified, for example from ovine brain using a combination of ion exchange and reversed phase HPLC techniques, analyzed by Syn-1 immunoblot and conventional Ag-stained SDS-PAGE analysis, and the NH₂-terminal Syn-1 protein sequenced. CNBr and trypsin p29 Syn-1 fragments may be resolved by e.g., microbore HPLC, and the fragments sequenced.

E. Cell Transfections and Synthesis of Proteins

DNA molecules composed of a protein gene or a portion thereof, can be operably linked into an expression vector and introduced into a host cell to enable the expression of these proteins by that cell. Alternatively, a protein may be cloned in viral hosts by introducing the “hybrid” gene operably linked to a promoter into the viral genome. The protein may then be expressed by replicating such a recombinant virus in a susceptible host.

A DNA sequence encoding a protein molecule may be recombined with vector DNA in accordance with conventional techniques. When expressing the protein molecule in a virus, the hybrid gene may be introduced into the viral genome by techniques well known in the art. For example, cloning in filamentous phage is generally described in Sambrook et al., supra. Thus, the present invention encompasses the expression of the desired proteins in either prokaryotic or eukaryotic cells, or viruses which replicate in prokaryotic or eukaryotic cells. Preferably, the proteins of the present invention are cloned and expressed in a virus.

Viral hosts for expression of the proteins of the present invention include viral particles which replicate in prokaryotic host or viral particles which infect and replicate in eukaryotic hosts. Preferably, viruses which replicate in prokaryotic host are used according to the present invention. Illustrative prokaryotic viruses include, but are not limited to, the Ff class of filamentous phage (fd, f1, M13, etc.), bacteriophage λ, bacteriophage T4, and bacteriophage T3. The preferred phage for expressing a protein is the Ff class of filamentous phage (including strains f1, fd and M13), as summarized by G. P. Smith (Current Opinion in Biotechnology, Vol. 2/5, October 1991). “Phage” as used herein also includes “phagemids,” which are plasmids containing DNA sequences that allow the plasmid DNA to be packaged into filamentous phage particles when the plasmid-bearing cell receives an appropriate helper genome (e.g. Mead and Kemper, in Vectors: A Survey of Molecular Cloning Vectors and Their Uses, edited by R. Rodriquez and D. T. Denhardt, Butterworths, 1987, pp. 103-111). Examples of eukaryotic viruses include adenovirus, bovine papilloma virus, simian virus, tobacco mosaic virus and the like.

Expression of the desired protein in a viral host may be accomplished by operably linking the “hybrid” gene encoding a protein to a viral promoter. Alternatively, a promoter from the host in which the virus replicates may also be used. Preferably, the viral surface protein promoter is used to express the protein in the viral host. For example, the DNA molecule encoding a binding molecule can be inserted directly at any location within the surface protein gene. As long as the insertion maintains the correct reading frame for both the binding molecule and the surface protein genes (or portions of these genes), it is possible to express the proteins of the present invention in a viral microorganism. As used herein, the term “binding molecule” refers to a protein or portion thereof which binds to another protein or portion thereof, which is usually termed a ligand.

The expression of the proteins of the present invention can also be accomplished in procaryotic cells. Preferred prokaryotic hosts include bacteria, such as E. coli, Bacillus, Streptomyces, Pseudomonas, Salmonella, Serratia. The most preferred prokaryotic host is E. coli.

To express the desired protein in a prokaryotic cell (such as, for example, E. coli, B. subtilis, Pseudomonas, Streptomyces, etc.), it is necessary to operably link the desired protein encoding sequence to a functional prokaryotic promoter. Such promoters may be either constitutive or regulatable (i.e., inducible or derepressible). Examples of constitutive promoters include the int promoter of bacteriophage λ, and the bla promoter of the β-lactamase gene of pBR322, etc. Examples of inducible prokaryotic promoters include the major right and left promoters of bacteriophage λ (P_{L} and P_{R}), the trp, recA, lacZ, lacI, gal, and tac promoters of E. coli, the a-amylase (Ulmanen et al., J. Bacteriol. 162:176-182 (1985)), and the s-28-specific promoters of B. subtilis (Gilman, et al., Gene 32:11-20 (1984)), the promoters of the bacteriophages of Bacillus (Gryczan, T. J., In: The Molecular Biology of the Bacilli, Academic Press, Inc., NY (1982)), and Streptomyces promoters (Ward et al., Mol. Gen. Genet. 203:468-478 (1986)). Prokaryotic promoters are reviewed by Glick, B. R., (J. Ind. Microbiol. 1:277-282 (1987)); Cenatiempo, Y. (Biochimie 68:505-516 (1986)); and Gottesman, S. (Ann. Rev. Genet. 18:415-442 (1984)). Proper expression in a prokaryotic cell also requires the presence of a ribosome binding site upstream from the protein-encoding sequence. Such ribosome binding sites are disclosed, for example, by Gold et al. (Ann. Rev. Microbiol. 35:365-404 (1981)).

Eukaryotic hosts for cloning and expression of the proteins of the present invention include insect cells, yeast (especially Saccharomyces), fungi (especially Aspergillus), and mammalian cells (such as, for example, human or primate cells) either in vivo, or in tissue culture.

The expression of the desired protein in eukaryotic hosts requires the use of eukaryotic regulatory regions. Such regions will, in general, include a promoter region sufficient to direct the initiation of RNA synthesis. Preferred eukaryotic promoters include the promoter of the mouse metallothionein I gene (Hamer et al., J. Mol. Appl. Gen. 1:273-288 (1982)); the TK promoter of Herpes virus (McKnight, S., Cell 31:355-365 (1982)); the SV40 early promoter (Benoist et al., Nature (London) 290:304-310 (1981)); the yeast gal4 gene promoter (Johnston et al., Proc. Natl. Acad. Sci. (USA) 79:6971-6975 (1982); Silver et al., Proc. Natl. Acad. Sci. (USA) 81:5951-5955 (1984)). As is widely known, translation of eukaryotic mRNA is initiated at the codon which encodes the first methionine. For this reason, it is preferable to ensure that the linkage between a eukaryotic promoter and a DNA sequence which encodes the desired protein does not contain any intervening codons which are capable of encoding a methionine (ie., AUG).

The desired protein encoding sequence and an operably linked promoter may be introduced into a recipient prokaryotic or eukaryotic cell either as a non-replicating DNA (or RNA) molecule, which may either be a linear molecule or, more preferably, a closed covalent circular molecule. Since such molecules are incapable of autonomous replication, the expression of the desired protein may occur through the transient expression of the introduced sequence. Alternatively, permanent expression may occur through the integration of the introduced sequence into the host chromosome. For expression of the desired protein in a virus, the hybrid gene operably linked to a promoter is typically integrated into the viral genome, be it RNA or DNA. Cloning into viruses is well known and thus one of skill in the art will know numerous techniques to accomplish such cloning.

Cells which have stably integrated the introduced DNA into their chromosomes can be selected by also introducing one or more markers which allow for selection of host cells which contain the expression vector. The marker may complement an auxotrophy in the host (such as leu2, or ura3, which are common yeast auxotrophic markers), biocide resistance, e.g., antibiotics, or resistance to heavy metals, such as copper, or the like. The selectable marker gene can either be directly linked to the DNA gene sequences to be expressed, or introduced into the same cell by co-transfection.

In another embodiment, the introduced sequence will be incorporated into a plasmid or viral vector capable of autonomous replication in the recipient host cell. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

For a mammalian host, several possible vector systems are available for expression. One class of vectors utilize DNA elements which provide autonomously replicating extra-chromosomal plasmids, derived from animal viruses such as bovine papilloma virus, polyoma virus, adenovirus, or SV40 virus. A second class of vectors relies upon the integration of the desired gene sequences into the host chromosome. Cells which have stably integrated the introduced DNA into their chromosomes may be selected by also introducing one or more markers which allow selection of host cells which contain the expression vector. The marker may provide for prototropy to an auxotrophic host, biocide resistance, e.g., antibiotics, or resistance to heavy metals, such as copper or the like. The selectable marker gene can either be directly linked to the DNA sequences to be expressed, or introduced into the same cell by co-transformation. Additional elements may also be needed for optimal synthesis of mRNA. These elements may include splice signals, as well as transcription promoters, enhancers, and termination signals. The cDNA expression vectors incorporating such elements include those described by Okayama, H., Mol. Cell. Biol. 3:280 (1983), and others.

Prokaryotic vectors used to express the proteins of the present invention include plasmids such as those capable of replication in E. coli such as, for example, PBR322, ColE1, pSC101, pACYC 184, πVX. Such plasmids are, for example, disclosed by Maniatis, T., et al. (In: Molecular Cloning, A Laboratory Manual, Cold Spring Harbor Press, Cold Spring Harbor, N.Y. (1982)). Bacillus plasmids include pC194, pC221, pT127, etc. Such plasmids are disclosed by Gryczan, T. (In: The Molecular Biology of the Bacilli, Academic Press, NY (1982), pp. 307329). Suitable Streptomyces plasmids include pIJ101 (Kendall et al., J. Bacteriol. 169:41774183 (1987)), and Streptomyces bacteriophages such as ØC31 (Chater et al., In: Sixth International Symposium on Actinomycetales Biology, Akademiai Kaido, Budapest, Hungary (1986), pp. 45-54). Pseudomonas plasmids are reviewed by John et al. (Rev. Infect. Dis. 8:693-704 (1986)), and Izaki, K. (Jpn. J. Bacteriol. 33:729-742 (1978)).

Once the vector or DNA sequence containing the constructs has been prepared for expression, the DNA constructs may be introduced or transformed into an appropriate host. Various techniques may be employed, such as protoplast fusion, calcium phosphate precipitation, electroporation, or other conventional techniques. As is well known, viral sequences containing the “hybrid” protein gene may be directly transformed into a susceptible host or first packaged into a viral particle and then introduced into a susceptible host by infection. After the cells have been transformed with the recombinant DNA (or RNA) molecule, or the virus or its genetic sequence is introduced into a susceptible host, the cells are grown in media and screened for appropriate activities. Expression of the sequence results in the production of the protein of the present invention.

Immunoprecipitation using FGF-1 and Syn-1 antibodies of recombinant FGF-1 Cu²⁺-induced homodimer (see, Engleka et al. (1992)) with recombinant p29 Syn-1 followed by FGF1/Syn-1 immunoblot analysis as a function of pS concentration, temperature, pH and ionic strength may be used to confirm the interaction between FGF-1 and/or FGF-1 homodimer and p29 Syn-1. Even if post-translational modifications of FGF-1/Syn-1 occur, sequencing of p29 Syn-1 will identify those residues that are post-translationally modified.

The p29 Syn-1 fragment, or any of the above-described fragments or genes may be expressed using known expression system, for example the Baculovirus, whereupon the p29 Syn-1 fragment would be expressed as a GST fusion protein. Fluorescence temperature spectroscopy studies of the FGF-1/p29 Syn-1 complex may also provide useful information relative to changes in protein conformation.

F. Regulation of FGF-1 export

### 1. Inhibitors/Enhancers

A variety of useful inhibitors and enhancers that inhibit or promote the export of FGF-1 are contemplated by the present invention. As one will observe from a review of FIG. 1 and the related Examples, a variety of compositions and constructs are useful in this regard.

One “class” of inhibitors includes compounds or compositions that associate with an enzyme, such as a protease, in such a manner as to inhibit the normal function of the enzyme. Such inhibition can be effected by a variety of ways, including binding of the inhibitor to a site on the enzyme such that the substrate binding site is blocked through stearic hindrance; binding of the inhibitor to the active site of the enzyme and thus preventing the substrate from having access to the active site; binding of the inhibitor to the enzyme in such a manner that changes the secondary or tertiary structure of the enzyme and therefore inhibits its activity (allosteric effects); disrupting or preventing the interaction of a required cofactor with the enzyme; and in other ways appreciated by those of skill in the art.

Another “class” of inhibitors includes compounds or compositions that prevent the interaction of one of the molecules in the FGF-1 export pathway with another molecule or with an intracellular vesicle (or component thereof), thereby disrupting the FGF-1 export pathway. For example, inhibitors that inhibit the formation of a complex comprising FGF-1, or inhibit the complexation of FGF-1 and Syn-1, are useful as described herein. Such inhibitors include compounds or compositions that bind to FGF-1, to Syn-1, to pS, or to any of the proteins in the FGF-1 export pathway, and prevent said protein(s) from interactions that are a prerequisite to the export of FGF-1. For example, an antibody (or immunologically active fragments thereof) which binds to Syn-1 in the cytosol, thereby making it unavailable for complexation with FGF-1, are also useful as disclosed herein.

Inhibitors which prevent or disrupt intracellular trafficking of any component of the FGF-1 export pathway (see, e.g., FIG. 1) also include compounds and compositions which inhibit or otherwise disrupt the expression of one or more proteins involved in the FGF-1 export pathway. Therefore, inhibitory agents which selectively interfere with the transcription and/or translation of genes that express the proteins involved in the FGF-1 export pathway are useful as disclosed herein, as are inhibitors which disrupt posttranslational processing and modification of said proteins.

Examples of such molecules include antisense nucleotides, triplex molecules, and ribozymes. Antisense molecules are oligonucleotides that bind in a sequence-specific manner to nucleic acids, such as mRNA or DNA. When bound to mRNA that has complementary sequences, antisense prevents translation of the mRNA (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. (1993) Nucl. Acids Res. 21:3405-3411, which describes dumbbell antisense oligonucleotides). Triplex molecules refer to single DNA strands that bind duplex DNA forming a colinear triplex molecule, thereby preventing transcription (see, e.g., U.S. Pat. No. 5,176,996 to Hogan et al., which describes methods for making synthetic oligonucleotides that bind to target sites on duplex DNA).

A ribozyme is an RNA molecule that specifically cleaves RNA or DNA substrates, such as mRNA, resulting in inhibition or interference with cell growth or expression. There are at least five known classes of ribozymes involved in the cleavage and/or ligation of RNA chains. Ribozymes can be targeted to any RNA transcript and can catalytically cleave such transcript (see, e.g., U.S. Pat. Nos. 5,272,262; 5,144,019; and s. 5,168,053, 5,180,818, 5,116,742 and 5,093,246 to Cech et al.). Any such ribozyme or nucleic acid encoding the ribozyme may be delivered to cells as disclosed herein.

Also, as disclosed herein, a number of proteins and related molecules (e.g., nucleotides, cofactors, etc.) are found in a cell that has been exposed to some type of stress. As described herein, stable complexes comprising FGF-1 and Syn-1 have been isolated and identified.

The involvement of other proteins in the formation of a complex comprising FGF-1 and Syn-1 is also evident, in vivo as well as in vitro. Some of these “other proteins” have also been found in complexes that include FGF-1 and Syn-1, as disclosed herein.

Tracing the putative pathway back from the eventual release of FGF-1 from the cell—which pathway is broadly represented by the diagram set forth in FIG. 1 (albeit understood that the invention is not limited to, or by, the putative pathway)—one can appreciate that inhibitors or enhancers affecting FGF-1 export or release from the cell can be effective at various points in the pathway. For instance, such inhibitors or enhancers—which are referred to collectively herein as “agents”—may disrupt or stimulate the release of FGF-1 from the cell membrane. Alternatively, such agents may act to disrupt or stimulate the protease or protease complex that catalyzes the release of FGF-1 from the FGF-1:Syn-1 complex.

Because cyclophilin B (CpB) is believed to interact with Syn-1, and since CpB is present in various complexes which also include FGF-1 and Syn-1, agents which stimulate or inhibit the interaction between CpB and Syn-1 may also up-regulate or down-regulate FGF-1 release. Moreover, since the order of assembly of the involved molecules into the complex may be a critical factor in addition to—or separate from—the physical presence of the molecule in the complex, any agent which interferes with or streamlines the assembly process may be an effective inhibitor or enhancer, as well.

Thus, any agent which interferes with the participation of CpB in the formation of a complex containing FGF-1 and/or Syn-1 is expected to have an up- or down-regulatory effect on FGF-1 export. Useful inhibitors would thus include molecules which bind or otherwise affect CpB availability, including the cyclosporins (particularly cyclosporin A), steroids, and other agents with anti-inflammatory effects. Molecules such as estrogen, hsp9o, cyclophilin A, and p59 may also be useful agents as described herein, as these molecules may form complexes with, or otherwise interact with CpB in a manner to make CpB unavailable (or less readily available) to participate in complexes comprising FGF-1.

Altering the amounts, availability and activities of calcineurin A, pS, and/or calmodulin, and modulating the calcium levels (e.g., the availability of Ca²⁺ ions to bind with pS/calmodulin) in the cell are expected to affect the association of molecules involved in FGF-1 release, whether those molecules are present in the final complex from which FGF-1 is released, or whether they are participants in intermediate events. Therefore, changes in the levels and interaction of the foregoing molecules is expected to stimulate or inhibit formation of the FGF-1-containing complex.

As disclosed herein, S100A13 is also involved in the putative pathway leading to FGF-1 release from the cell. Thus, any agent which interferes with or enhances the assembly of complexes containing S100A13 is expected to inhibit or stimulate FGF-1 export. Similarly, agents which disrupt or enhance the interaction of S100A13 with precursors in the series of interactions leading to the formation of complexes containing FGF-1 and/or Syn-1 will be useful.

Finally, agents which disrupt or enhance the interaction of Syn-1 with the secretory vesicle, or with the ability of Syn-1 to complex with FGF-1 or any other molecule involved in FGF-1-containing complexes, would be useful for the purposes disclosed herein. Logically, any agent which disrupts or stabilizes the conformation of any molecule involved in the FGF-1 containing complex would have an inhibitory or stimulatory effect, respectively.

2. Assays for Screening Inhibitors/Enhancers

Inhibitors of the secretion or export of leaderless proteins—and FGF-1 is described herein as exemplary, particularly within the context of the present invention—may be identified by an assay, such as the assays described herein. Briefly, in a preferred assay, a cell expressing FGF-1 or another leaderless protein (e.g. an FGF-1 mutein or a biologically active fragment thereof) is treated with the candidate inhibitor and the amount of FGF-1 detected as an extracellular protein is compared to the amount detected without treatment.

In any of the following assays (which are intended to be exemplary and do not constitute an exhaustive list), a compound inhibits export if there is a statistically significant reduction in the amount of protein detected extracellularly in the assay performed with the inhibitor compared to the assay performed without the inhibitor. Preferably, the inhibitor reduces export of the protein of interest by at least 50%, even more preferably 80% or greater, and also preferably, in a dose-dependent manner. In addition, there should be no statistically significant effect on the appearance of either the secreted protein or the cytosolic protein. Preferably, there is less than a 10% increase or decrease in the appearance of these two proteins.

Similarly, in any of these assays, a compound stimulates or enhances export if there is a statistically significant increase in the amount of protein detected extracellularly in the assay performed with the enhancer compared to the assay performed without the enhancer. Preferably, the enhancer increases export of the protein of interest by at least 50%, even more preferably 80% or greater, and also preferably, in a dose-dependent manner. In addition, there should be no statistically significant effect on the appearance of either the secreted protein or the cytosolic protein. Preferably, there is less than a 10% increase or decrease in the appearance of these two proteins.

Within the context of the present invention, therefore, an inhibitor must satisfy various criteria. First, it must inhibit the export of FGF-1. Second, it must not inhibit export of a representative secreted protein with a leader sequence. Third, it must not promote expression of a representative cytosolic protein in the extracellular environment. Once inhibitors satisfying these requirements are identified, the utilization of assay procedures to identify the manner in which the export of FGF-1 is inhibited are particularly useful.

Thus, inhibitors of FGF-1 export include various substances (e.g. therapeutic agents) which interfere with, inhibit or prevent (a) the formation of an FGF-1:Syn-1 complex; (b) the formation of a complex comprising (or including) FGF-1, Syn-1, or both; (c) the formation of a complex comprising FGF-1, Syn-1, cyclophilin-B, or any combination thereof; and (d) the formation of a complex comprising FGF-1, Syn-1, cyclophilin-B, S100A13, or any combination thereof. Inhibitors of FGF-1 export also include therapeutic agents which interfere with, inhibit or prevent (e) the release of FGF-1 from a complex including any one or more of the components recited in (a)-(d) above; or (f) trafficking of a complex comprising any one or more of the components recited in (a)-(d) above to the cell membrane.

Inhibitors of FGF-1 export contemplated within the scope of the present invention also include therapeutic agents which inhibit the trafficking of FGF-1 to the cell membrane by interfering with other mechanisms.

As disclosed herein, the present invention also contemplates the identification and use of therapeutic agents which stimulate or enhance the secretion of FGF—and more particularly, FGF-1. Thus, in a preferred assay, a cell expressing FGF-1 or another leaderless protein (e.g. an FGF-1 mutein or a biologically active fragment thereof) is treated with the candidate enhancing or up-regulating agent and the amount of FGF-1 detected as an extracellular protein is compared to the amount detected without treatment.

An enhancer must satisfy various criteria, within the context of the present invention. First, it must enhance the export of FGF-1. Second, it must not enhance export of a representative secreted protein with a leader sequence. Third, it must not promote expression of a representative cytosolic protein in the extracellular environment. Once enhancers satisfying these requirements are identified, the utilization of assay procedures to identify the manner in which the export of FGF-1 is enhanced are particularly useful.

Thus, enhancers of FGF-1 export include various substances (e.g. therapeutic agents) which stimulate or enhance (a) the formation of an FGF-1:Syn-1 complex; (b) the formation of a complex comprising (or including) FGF-1, Syn-1, or both; (c) the formation of a complex comprising FGF-1, Syn-1, cyclophilin-B, or any combination thereof; and (d) the formation of a complex comprising FGF-1, Syn-1, cyclophilin-B, S100A13, or any combination thereof. Enhancers of FGF-1 export also include therapeutic agents which enhance (e) the release of FGF-1 from a complex including any one or more of the components recited in (a)-(d) above; or (f) trafficking of a complex comprising any one or more of the components recited in (a)-(d) above to the cell membrane.

Enhancers of FGF-1 export contemplated within the scope of the present invention also include therapeutic agents which stimulate or increase the trafficking of FGF-1 to the cell membrane by enhancing or up-regulating other mechanisms.

Assays to detect leaderless protein, secreted protein, and cytosolic protein in a cell-based assay include immunoprecipitation of proteins labeled in a pulse-chase procedure, ELISA, 2-D gels, protein stains (e.g., Coomassie blue), HPLC, Western Blot, biological assays, and phagokinetic tracts. Such assays may be employed in the identification of useful up- or down-regulating therapeutic agents. In all these assays, test cells expressing and exporting a leaderless protein (e.g., FGF-1) or other protein of interest are incubated with and without the candidate inhibitor or enhancer.

Immunoprecipitation is an assay that may be used to determine inhibition or enhancement of the release/production of FGF-1 or any other protein(s) involved in the aforementioned complexes. Briefly, using FGF-1 as an example, cells that naturally express FGF-1 (or a biologically active fragment or mutein thereof) or express FGF-1 or a variant thereof from an introduced vector construct are labeled with ³⁵S-methionine and/or ³⁵S-cysteine for a brief period of time, typically 15 minutes or longer, in methionine- and/or cysteine-free cell culture medium. Following pulse-labeling, cells are washed with medium supplemented with excess unlabeled methionine and cysteine plus heparin if the leaderless protein is heparin-binding. Cells are then cultured in the same chase medium for various periods of time.

Candidate inhibitors or enhancers are added to cultures at various concentration. Culture supernatant is collected and clarified. Supernatants are incubated with anti-FGF-1 immune serum or a monoclonal antibody, or with anti-tag antibody if a peptide tag is present, followed by a developing reagent such as Staphylococcus aureus Cowan strain I, protein A-Sepharose®, or Gamma-bind™ G-Sepharose®. (See, e.g., U.S. Pat. No. 5,437,995, which discloses an FGF-1 antibody useful as described herein. The disclosures of said patent are incorporated herein by reference.)

Immune complexes are pelleted by centrifugation, washed in a buffer containing 1% NP-40 and 0.5% deoxycholate, EGTA, PMSF, aprotinin, leupeptin, and pepstatin. Precipitates are then washed in a buffer containing sodium phosphate pH 7.2, deoxycholate, NP-40, and SDS. Immune complexes are eluted into an SDS gel sample buffer and separated by SDS-PAGE. The gel is processed for fluorography, dried, and exposed to x-ray film.

Alternatively, ELISA may be used to detect and quantify the amount of FGF-1 (or other protein(s) involved in the complexes noted herein) present in cell supernatants. ELISA is preferred for detection in high throughput screening. Briefly, when FGF-1 is the “test protein,” 96-well plates are coated with an FGF-1 antibody or anti-tag antibody, washed, and blocked with 2% BSA. Cell supernatant is then added to the wells. Following incubation and washing, a second antibody (e.g., to FGF-1) is added. The second antibody may be coupled to a label or detecting reagent, such as an enzyme or to biotin.

Following further incubation, a developing reagent is added and the amount of FGF-1 determined using an ELISA plate reader. The developing reagent is a substrate for the enzyme coupled to the second antibody (typically an anti-isotype antibody) or for the enzyme coupled to streptavidin. Suitable enzymes are well known in the art and include horseradish peroxidase, which acts upon a substrate (e.g., ABTS) resulting in a colorimetric reaction. It will be appreciated that rather than using a second antibody coupled to an enzyme, the FGF-1 antibody may be directly coupled to the horseradish peroxidase, or other equivalent detection reagent. If necessary, cell supernatants may be concentrated to raise the detection level.

ELISA may also be readily adapted for screening multiple candidate inhibitors or enhancers with high throughput. Briefly, such an assay is conveniently cell-based and performed in 96-well plates. If test cells naturally or stably express the protein of interest—e.g., FGF-1—the cells are plated at 20,000 cells/well. If the cells do not naturally express the protein, transient expression is achieved, such as by electroporation or Ca₂PO₄-mediated transfection.

For electroporation, an exemplary assay would comprise dispensing 100 μl of a mixture of cells (150,000 cells/ml) and vector DNA (5 μg/ml) into individual wells of a 96-well plate. The cells are electroporated using an apparatus with a 96-well electrode (e.g., ECM 600 Electroporation System, BTX, Genetronics, Inc.). Optimal conditions for electroporation are experimentally determined for the particular host cell type. Voltage, resistance, and pulse length are the typical parameters varied. Guidelines for optimizing electroporation may be obtained from manufacturers or found in protocol manuals, such as Current Protocols in Molecular Biology (Ausubel et al. (ed.), Wiley Interscience, 1987).

Cells are diluted with an equal volume of medium and incubated for 48 hours. Electroporation may be performed on various cell types, including mammalian cells, yeast cells, bacteria, and the like. Following incubation, medium with or without inhibitor or enhancer is added and cells are further incubated for 1-2 days. At this time, culture medium is harvested and the protein is assayed by any of the assays herein. Preferably, ELISA is used to detect the protein. An initial concentration of 50 μM is tested. If this amount gives a statistically significant reduction or increase of export, the candidate inhibitor or enhancer is further tested in a dose response.

Alternatively, concentrated supernatant may be electrophoresed on an SDS-PAGE gel and transferred to a solid support, such as nylon or nitrocellulose. The protein of interest is then detected by an immunoblot (see, e.g., Harlow, Antibodies: A Laboratory Manual, Cold Spring Harbor Laboratory, 1988), using an antibody to the protein of interest containing an isotopic or non-isotopic reporter group. These reporter groups include, but are not limited to enzymes, cofactors, dyes, radioisotopes, luminescent molecules, fluorescent molecules and biotin. Preferably, the reporter group is ¹²⁵I or horseradish peroxidase, which may be detected by incubation with 2,2′-azino-di-3-ethylbenzthiazoline sulfonic acid. These detection assays described above are readily adapted for use if the protein of interest contains a peptide tag. In such case, the antibody used will bind to the peptide tag. Other assays include size or charge-based chromatography, including HPLC, and affinity chromatography.

Other proteins of interest (e.g., FGF-2, PD-ECGF) may be assessed in similar manner. Proteins of interest displaying other functions may be assayed in other appropriate bioassays available in the art.

Other in vitro angiogenic assays include measuring proliferation of endothelial cells within collagen gel (Goto et al., Lab Invest. 69:508, 1993), co-culture of brain capillary endothelial cells on collagen gels separated by a chamber from cells exporting the protein of interest (Okamure et al., B.B.R.C. 186:1471, 1992; Abe et al., J. Clin. Invest. 92:54, 1993), or a cell migration assay (see, Warren et al., J. Clin. Invest. 95:1789, 1995).

Alternatively, or as a further assessment of candidate inhibitors, inhibition assays may be performed by assaying the extent of binding between a protein of interest and its transport protein or proteins. A host cell expressing both the protein of interest and transport protein endogenously or following transfection are treated with candidate inhibitors. The binding may be measured by a variety of different methods. A co-precipitation assay in which antibodies to either protein are used to precipitate the preselected proteins may be used. The precipitates are assayed by gel electrophoresis for disruption of the interaction. The protein of interest may be a fusion protein with a tag peptide and anti-tag peptide antibodies are used for precipitation. As described above, the membrane bound α1 subunit or fragment may be used in this assay.

An assay for identifying an inhibitor or enhancer of export may be performed using isolated transport molecule(s) and the protein of interest. Isolated components are preferably obtained by recombinant expression and purified by standard methodologies. In such an assay, the isolated components are mixed, along with any necessary cofactors, in the presence or absence of the candidate inhibitor or enhancer. The extent of binding of the protein of interest and transport molecule is measured. This assay may conveniently be performed in an ELISA or ELISA-style format.

Briefly, using inhibitors as exemplary, the transport molecule is adhered to the wells of a 96-well plate. The protein of interest with or without candidate inhibitors is added to the wells and incubated. Unbound protein is washed away, and the protein of interest is detected by labeled antibody as described herein, for example. Variations on this assay may be used. For example, the components may be attached to Biocore chips or similar solid phase detection device.

The activity of a therapeutic agent of interest (e.g., inhibitor or enhancer) may also be measured by in vivo models of disease. For example, a cell that exports the protein of interest to the extracellular environment is introduced into a local milieu where the activity of the protein can be measured. In the case of a cell that exports FGF-1, for example, the cell will promote vascularization or angiogenesis, inflammation, clotting, or gliosis on neighboring cells. For example, in the case of a cell exporting FGF-1 that is inoculated along with tumor cells, vascularization of the tumor will ensue. Accordingly, an inhibitor of FGF-1 export will inhibit growth of the tumor. One skilled in the art will recognize that the export levels of the protein may be varied through the use of promoters of varying strength. In addition, cells exporting the protein may be transformed stably or express the protein transiently. The site and route of administration depends in part upon the protein and its normal site of action.

For proteins of interest that cause cell motility, such s FGF, a phagokinetic tract assay may be used to determine the amount of protein of interest exported (Mignatti et al., J. Cellular Physiol. 151:81-93, 1992). In this assay, cells are allowed to migrate on a microscope cover slip coated with colloidal gold. Under dark field illumination, the gold particles appear as a homogenous layer of highly refringent particles on a dark background. When a cell migrates on the substrate, it pushes aside the gold particles producing a dark track. An image analyzer may be used to measure the length of the tracks. Under predetermined, standard conditions, cell motility directly correlates with the amount of FGF-2 produced by the cells. The choice of the bioassay will depend, at least in part, by the protein of interest tested.

In vivo assays may be used to confirm that an inhibitor or enhancer affects export of protein of interest. For measuring angiogenic activity, standard assays include the chicken chorioallantoic membrane assay (Aurbach et al., Dev. Biol. 41:391, 1974; Taylor and Folkman, Nature 247:307, 1982) and inhibition of angiogenesis in tumors. For some proteins of interest, an assay measuring inhibition of tumor growth, such as in a murine xenogeneic tumor model, may be appropriate.

F. Administration

As described above, FGF-1 is useful for repairing wounded, injured and/or damaged tissue or organs, among other uses. Repair or treatment of means that symptoms are lessened, or the progression of the disease or conditions are halted or delayed, or wounded, injured or damaged tissue or organs are made measurably or noticeably less wounded, injured or damaged as determined by at least one criterion, or healed. Therefore, agents useful for the enhancement or stimulation of secretion and/or export of FGF-1 into the bloodstream are advantageous for accelerating, repairing or healing wounded, injured and or damaged tissue.

In contrast, the over expression of FGF-1 has been associated with the uncontrolled production of tissue, such as occurs in the initial development or growth of tumors, including cancerous tumors. Consequently, agents useful for inhibiting, reducing or abolishing the secretion and/or export of FGF-1 into the bloodstream are advantageous for inhibiting, reducing, or halting the growth of a particular class of unwanted tissue, including tumors.

By example, cells to be treated are contacted with an inhibitor or an enhancer at a therapeutically effective dosage. Contacting may be effected by incubation of cells ex vivo or in vivo, such as by topical treatment, delivery by specific carrier, or by vascular supply.

Similarly, therapeutic agents which up-regulate, stimulate or otherwise facilitate the secretion of FGF-1 are also useful for ameliorating a variety of conditions, including repair situations, such as the repair of spinal cord, nerve or ulcerative damage, injury or degeneration.

The inhibitors or enhancers disclosed herein may be formulated into pharmaceutical compositions suitable for topical, local, intravenous and systemic application. Time release formulations are also desirable. Effective concentrations of one or more of the conjugates are mixed with a suitable pharmaceutical carrier or vehicle. The concentrations or amounts of the conjugates that are effective requires delivery of an amount, upon administration, that ameliorates the symptoms or treats the disease. Typically, the compositions are formulated for single dosage administration.

Therapeutically effective concentrations and amounts may be determined empirically by testing the therapeutic agents or substances of the present invention in known in vitro and in vivo systems, such as those described herein; dosages for humans or other animals may then be extrapolated therefrom.

Candidate tumors for treatment as described herein include those with receptors for FGF. Such tumors include, but are not limited to, melanomas, teratocarcinomas, ovarian carcinomas, bladder tumors, and neuroblastomas. Other diseases, disorders, and syndromes are suitable for treatment and include rheumatoid arthritis, diabetic retinopathy, psoriasis, and the like.

Pharmaceutical carriers or vehicles suitable for administration of the therapeutic agents provided herein include any such carriers known to those skilled in the art to be suitable for the particular mode of administration. In addition, the agents may be formulated as the sole pharmaceutically active ingredient in the composition or may be combined with other active ingredients.

The compositions of the present invention may be prepared for administration by a variety of different routes. Local administration is preferred. The therapeutic agent may be mixed with suitable excipients, such as salts, buffers, stabilizers, and the like. If applied topically, such as to the skin and mucous membranes, the agent may be in the form of gels, creams, and lotions. Such solutions, particularly those intended for ophthalmic use, may be formulated as, for example, 0.01%-10% isotonic solutions, pH about 5-7, with appropriate salts (see, e.g., U.S. Pat. No. 5,116,868).

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of toxicity such as sodium chloride or dextrose. Parenteral preparations can be enclosed in ampules, disposable syringes or multiple dose vials made of glass, plastic or other suitable material.

If therapeutic agents of the present invention are administered intravenously, suitable carriers include physiological saline or phosphate buffered saline (PBS), and solutions containing thickening and solubilizing agents, such as glucose, polyethylene glycol, and polypropylene glycol and mixtures thereof. Liposomal suspensions may also be suitable as pharmaceutically acceptable carriers. These may be prepared according to methods known to those skilled in the art.

The therapeutic agent may be prepared with carriers that protect it against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others. For example, the composition may be applied during surgery using a sponge, such as a commercially available surgical sponge (see, e.g., U.S. Pat. Nos. 3,956,044 and 4,045,238).

The agents or substances of the invention can be administered by any appropriate route, for example, orally, parenterally, intravenously, intradermally, subcutaneously, or topically, in liquid, semi-liquid or solid form and are formulated in a manner suitable for each route of administration. Preferred modes of administration depend upon the indication treated. Dermatological and ophthalmologic indications will typically be treated locally; whereas, tumors, restenosis, and infections will typically be treated by systemic, intradermal or intramuscular modes of administration.

The therapeutic agent is included in the pharmaceutically acceptable carrier in an amount sufficient to exert a therapeutically useful effect in the absence of undesirable side effects. It is understood that number and degree of side effects depends upon the condition for which the agents are administered. For example, certain toxic and undesirable side effects are tolerated when treating life-threatening illnesses, such as tumors, that would not be tolerated when treating disorders of lesser consequence. The concentration of therapeutic agent in the composition will depend on absorption, inactivation and excretion rates thereof, the dosage schedule, and amount administered, as well as other factors known to those of skill in the art.

The therapeutic agent may be administered one time, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

G. FGF Constructs.

Constructs expressing FGF alone or as a fusion protein are advantageous for use as described in the Examples that follow. The production of sufficient quantities of expression product (e.g., recombinant variants of FGF-1) for use in the various model systems and assays as disclosed herein advantageously employs a variety of known cell lines and vectors.

For example, U.S. Pat. No. 5,395,756 describes the production of acidic FGF protein (FGF-1), using an expression vector containing a cDNA sequence encoding a human acidic FGF protein with a T7 promoter upstream therefrom. Vectors including a sequence encoding a deletion-type mutein lacking up to 43 amino acids at the N-terminus are also disclosed. Accordingly, vectors useful in the transformation of host cells are available in the art, as are methods for transfecting same to permit the resultant expression of the gene/gene product of interest.

There are also advantages to maintaining the cys30 residue and to deleting the first 20 amino acid residues. Cys30 enables FGF-1 to subsequently utilize its COOH-terminal domain (residues 112-138) to associate with pS in an unfolded state at 42° C. Because the ps-binding domain in FGF-1 coincides with the domain responsible for cytosolic retention of the intracellular FGF-1 translation product as a non-nuclear, cytosolic protein, FGF-1: β-gal mutants may be used to define the boundaries of this domain required for FGF-1₁₋₁₅₄ secretion.

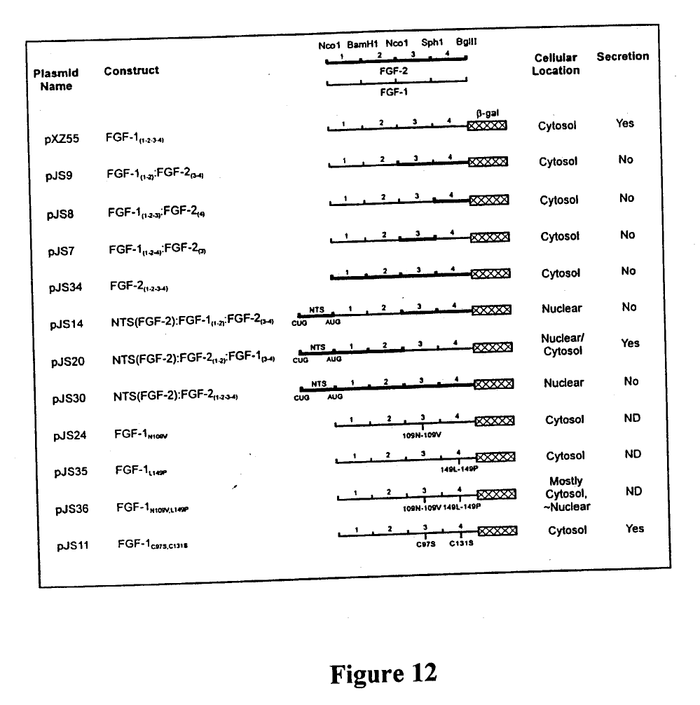

A number of synthetic FGF-1 cDNAs are available to construct a variety of FGF-1:FGF-2 cassette mutants, each containing the reporter gene, β-gal and stable NIH 3T3 cells. A list of exemplary chimera and point mutant FGF-1:β-gal chimera, each of which may be obtained, is shown in FIG. 12. The intracellular traffic of these chimeric proteins has been studied in detail using confocal β-gal immunofluorescence, X-gal staining and β-gal immunoblot analysis of cytosol and nuclear fractions prepared as previously described (see, Zhan et al. (1992)). These studies also suggest that the domain in FGF-1 responsible for cytosolic retention which enables FGF-1 to access the FGF-1 export pathway is not present in FGF-2 (see, FIG. 12). Moreover, FGF-2:β-gal is not exported in response to temperature stress (data not shown) under conditions identical to those described for the data in FIG. 12. These data also confirm the premise that nuclear-associated FGF-1 is not accessible to the FGF-1 export pathway.

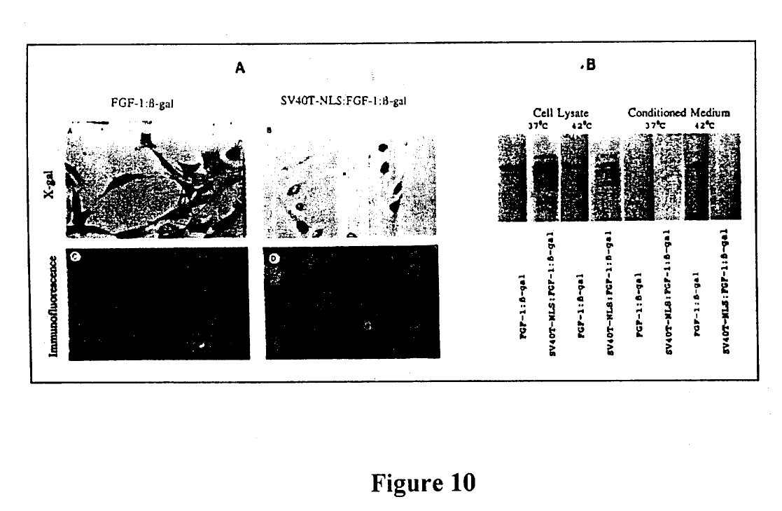

FIG. 12 shows FGF-1 mutants and chimeric constructs prepared by recombinant circle PCR (RC-PCR; see Friedman, et al., Biochem. Biophys. Res. Commun. 198: 1203-8 (1994)). Stable 3T3 cell transfectants for each of these constructs have been isolated, and the cytosolic level of the individual FGF-I₂₁₋₁₅₄ mutant/chimeric proteins are similar as measured by FGF-1 immunoblot analysis. The traffic of the individual constructs are shown; these data are derived from cell fractionation/immunoblot analysis, X-gal staining and β-gal immunohistochemical analysis as described in FIG. 10. Export (mislabeled on the drawing as “secretion”) of the individual construct was measured as described in FIG. 10. NTS=Nuclear Translocation Signal.

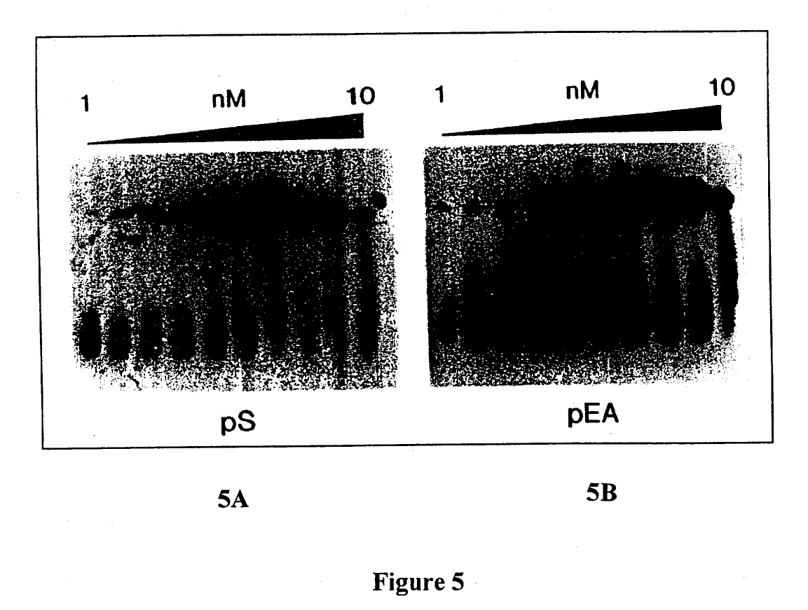

Of particular interest is the secretion of the N109 point mutant (FIG. 12), which exhibits cytosolic localization. This mutant is of interest because the FGF-1 crystalline structure (Zhu et al., Science 251:90-93 (1991)) predicts that residue N109 may be involved in the formation of a hydrogen bond with residue K124. Thus, if a mutant such as the FGF-1 N109: β-gal mutant is not exported in response to temperature stress as proposed, the residue can be mutated in full length FGF-1₁₋₁₅₄:β-gal (I) to determine the cytosol/nuclear locale of the chimera using the immunoblot/cell fractionation, X-gal staining and confocal immunofluorescence microscopy strategies as described (Zhan et al. (1992); Savion et al., (1994)); (ii) to determine whether the chimera is accessible to the heat shock-induced FGF-1 export pathway using conventional FGF-1 and/or β-gal immunoblot analysis of (NH₄)₂SO₄ and DTT activated conditioned medium as described (Jackson et al. (1992); Jackson et al. (1995); Tarantini et al. (1995)); and (iii) to determine not only whether the N109 point mutant is biologically active as a mitogen using established biological growth and DNA synthesis assays (e.g., Maciag et al. 1981 and 1984), but also whether the N109 point mutant is able to bind specifically to pS using the solid phase-binding (Tarantini et al. (1995)) and native gel shift assays as described in Example 3 and FIG. 5.

For example, wild-type FGF-1₁₋₁₅₄ translation is relatively poor in both conventional prokaryotic and eukaryotic expression systems. This poor translational efficiency of FGF-1 may be related to the presence of an exaggerated stem-loop structure with the FGF-1₁₋₁₅₄ mRNA, which was predicted by computer modeling (Forough et al., Biochem. Biophys. Acta 1090: 293-298 (1991)). The sequence of DNA encoding human FGF-1 has been reported in Jaye et al., Science 233:541-545, 1986; also see U.S. Pat. No. 5,223,483. It is also anticipated that muteins of FGF-1 will be useful in the within-disclosed compositions, complexes and according to the methods disclosed herein (see, e.g., U.S. Pat. No. 5,223,483).

Interestingly, mammalian cells are known to constitutively express reasonable levels of the FGF-1 transcript containing relatively small levels of the FGF-1 protein (Libermann et al., EMBO J. 6: 1627-1632 (1987)). However, prior to the present invention, it has been difficult to study FGF-1 export using these cell types in vitro.

In order to circumvent the apparent inefficient translation of the FGF-1₁₋₁₅₄ mRNA, a synthetic gene encoding FGF-₂₁₋₁₅₄ was prepared, in which the putative stem-loop mRNA structures have been minimized, especially the exaggerated stem structure which may result from the hybridization of the 3′-and 5′-ends of the FGF-1₁₋₁₅₄ ORF (see, Forough et al., (1991)). All of the functional (heparin-binding, receptor-binding) and biological (induction of cell migration, growth, DNA synthesis, angiogenesis, and neurotrophic behavior) features in vitro and in vivo are conserved in FGF-1₂₁₋₁₅₄ and are indistinguishable from those observed with FGF-11 ₁₅₄ (Burgess et al., J. Biol. Chem. 260:11389-11392 (1985)).

Moreover, expression of FGF-1₂₁₋₁₅₄ with or without the FGF-4₍ₛₛ₎ leader from stable transfectants (using the pMEXneo expression vector) in mammalian cells yields sufficient functional protein which is readily visualized using conventional heparin adsorption and immunologic procedures. The FGF-4 signal or leader sequence (FGF-4₍ₛₛ₎ is useful in the production of soluble FGF-1—i.e., an FGF-1 molecule including FGF-4₍ₛₛ₎ is forcibly transported out of the cell. NIH 3T3 cells have proven to be useful in the production of FGF-1 molecules when transfected with FGF-1_{21-154.}

Other deletions of the FGF-1 gene may accomplish high expression of a functional FGF-1 protein, so long as regions encoding critical functional and biological features of the protein are conserved to retain the in vitro and in vivo function of the molecule. Key attributes of the molecule appear to include, for example, both the cys30 residue and the COOH-terminal domain (residues 112-138), as disclosed below in sections addressing the construction of the FGF-1 molecule.

In one embodiment of the invention, the FGF-1₁₋₁₅₄ N109 construct is expressed using the pET3c expression vector, and the recombinant protein purified using either heparin and/or Cu²⁺ Sepharose affinity and/or reversed phase and/or ion exchange HPLC methods. The FGF-1₁₋₁₅₄ N109 mutant will traffic to the cytosol, but will not be exported in response to heat shock and will associate poorly, if at all, with pS. Similar studies with FGF-1₁₋₁₅₄ K124:β-gal will confirm these data.

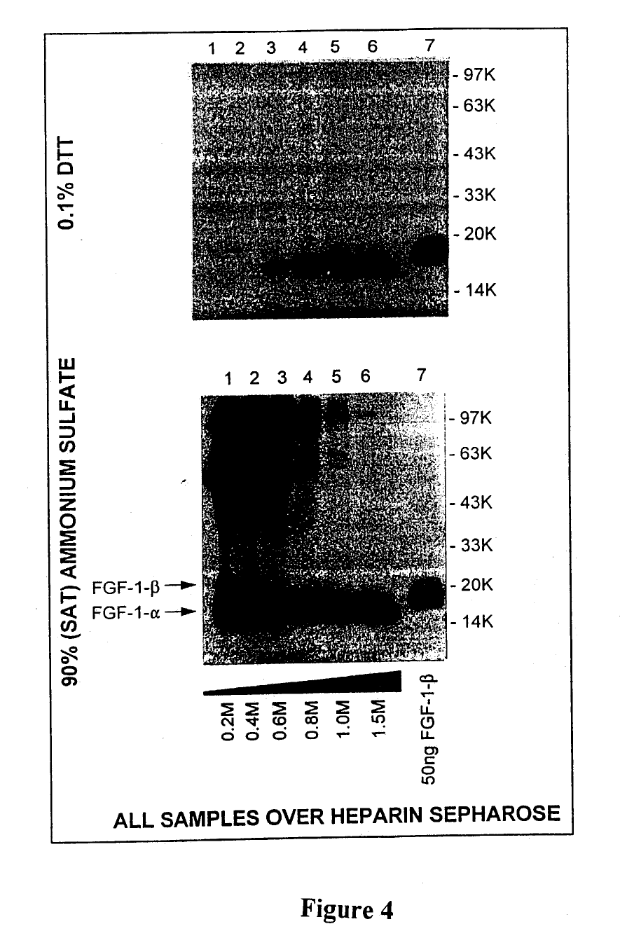

Another embodiment demonstrates that the model pathway established for FGF-1 export (FIG. 1), using FGF-1 lacking the NH₂-terminal residues 1-20 also applies to the full length FGF-1₁₋₁₅₄ translation product. The endogenous FGF-1₁₋₁₁₅₄ protein is observed in the conditioned medium in response to temperature stress (FIG. 4). Consequently, the FGF-1₂₁₋₁₅₄ construct was originally used so that it could be distinguished from the endogenous FGF-1₁₋₁₅₄ and the transfected FGF-1₂₁₋₁₅₄ proteins. Both forms of FGF-1 are indistinguishable in terms of their abilities to modify the cell migration and growth of a wide variety of target cells both in vitro and in vivo. Moreover, the expression of FGF-1₁₋₁₅₄ in the NIH 3T3 cell is only approximately 60% lower than the expression of the FGF-1₂₁₋₁₅₄ protein. Thus, secretion of FGF-1₁₅₄ should be reportable. However, even if it were not, an FGF-1₁₋₁₅₄:β-gal chimera could be readily constructed to study the secretion of the chimera after obtaining stable NIH 3T3 cell transfectants using increased sensitivity afforded by the enzymatic activity of the reporter gene as previously described (Zhan et al. (1992); Savion et al., (1994)).

Although these experiments have been exemplified in NIH 3T3 cells, the proposed mechanisms are expected to be applicable to other cell types. Thus, for example, endothelial cells (EC) and vascular smooth muscle cells (SMC) may be transfected with the FGF-1₁₋₁₅₄:β-gal chimera using the pMEXneo or other mammalian expression plasmid. Moreover, the exemplified use of the FGF-1₁₋₁₅₄:β-gal chimera permits the evaluation of the release of both the chimera and the endogenous form of FGF-1 since immunoblot analysis will resolve these proteins as Mᵣ 118 kDa and 20 kDa proteins.

As demonstrated in the NIH 3T3 cell, extracellular markers, such as lactate dehydrogenase, may be used to indicate cell lysis and to identify stable vascular cell transfectants expressing β-gal, IL-1αand FGF-2 for use as alternative controls. When stable β-gal, IL-1α and FGF-2 NIH 3T3 cell transfectants have been utilized, none of these proteins were released in response to temperature stress (data not shown). Thus, the model FGF-1 export pathway is generally applicable to numerous cell lines as well, including, but not limited to, HUVEC-ECV cells, murine LE-II cells, murine adipose EC and various rat SMCs. As those of skill in the relevant art will appreciate, there may be some differences between the various cell lines; however, such differences do not exceed the scope of the present invention.

As described in more detail herein, the levels of extracellular FGF-1 release in response to heat shock can be accurately quantitated by methods routinely used in the art, such as ELISAs. The stability of the recombinant FGF-1 protein, as well as the potential interference of endogenous fluids with the FGF-1 protein are readily determined prior to beginning the ELISA measurement for in vivo correlates. Control of other variables is within the routine practice of one of ordinary skill in the art.

In order that those skilled in the art can more fully understand this invention, the following examples are set forth. These examples are included solely for the purpose of illustration, and various modifications may be made without deviating from the spirit and scope of the invention. Accordingly, the invention is not limited except as by the appended claims.

### EXAMPLES

In the following examples and protocols, restriction enzymes, ligase, labels, and commercially available reagents were utilized in accordance with the manufacturer's recommendations. Standard methods and techniques for cloning, isolation, purification, labeling, and the like, as well as the preparation of standard reagents were performed essentially in accordance with Molecular Cloning: A Laboratory Manual, second edition, edited by Sambrook, Fritsch & Maniatis, Cold Spring Harbor Laboratory, 1989, and the revised third edition thereof.

Antibodies are essential to many of the following experiments. Rabbits are preferred for production of polyclonal antibodies. Procedurally, six rabbits are injected intradermally or intramuscularly with GST fusion or MAP peptides suspended in Freund's Complete Adjuvant (1:1), and bled in accordance with standard procedures. Booster injections are administered as necessary to maintain optimum antibody titer.

### Example 1

### Characterization of P45 Syn-1 as a Heparin- and Phosphatidylserine (pS)-Binding Protein

The serendipitous observations of Perin et al. (1991) led the present inventors to examine the conditioned medium of heat-shocked FGF-1 NIH 3T3 transfectants for the presence of Syn-1. Medium conditioned by heat shock (2 hr., 42° C.), as well as conditioned media (2 hr., 37° C.) was obtained from FGF-1 NIH 3T3 cell transfectants, activated by the addition of (NH₄)₂S0₄ and absorbed/eluted from heparin Sepharose. An immunoblot analysis of each sample was performed using a Syn-1 antibody. The antibodies to Syn-1 and to FGF-1 used and discussed throughout the present experiments were produced in the inventors' laboratories through the use of a combination of synthetic or recombinant Syn-1 and/or FGF-1 protein molecules. Recombinant p45 Syn-1 (100 ng in lane 3) served as a positive control.

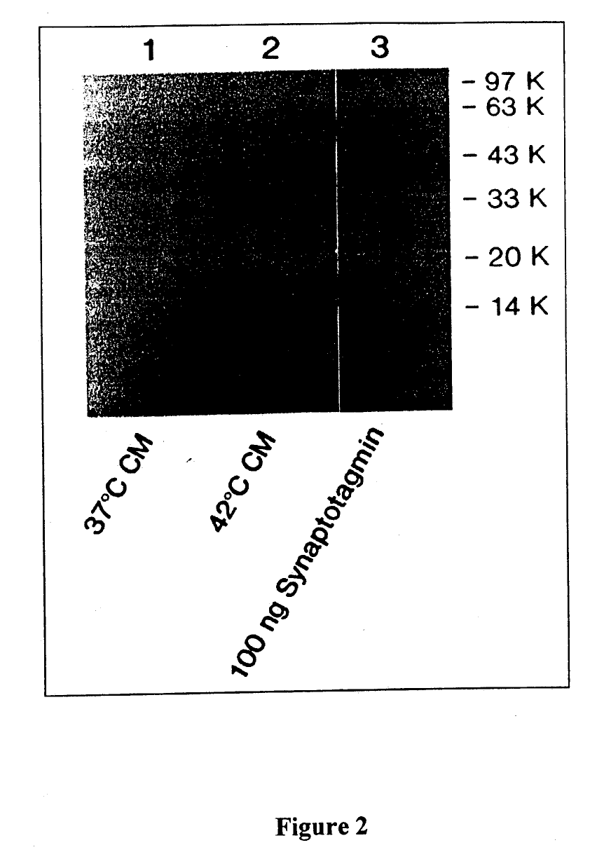

As shown in FIG. 2, by comparing lane 2 with the control media, a Syn-1 fragment was identified as a p40 heparin-binding protein. The mobility of the protein is interesting in that the recombinant p45 Syn-1 has an apparent M, 42 kDa, while the Syn-1 in the temperature conditioned media has an apparent Mᵣ˜40 kDa.

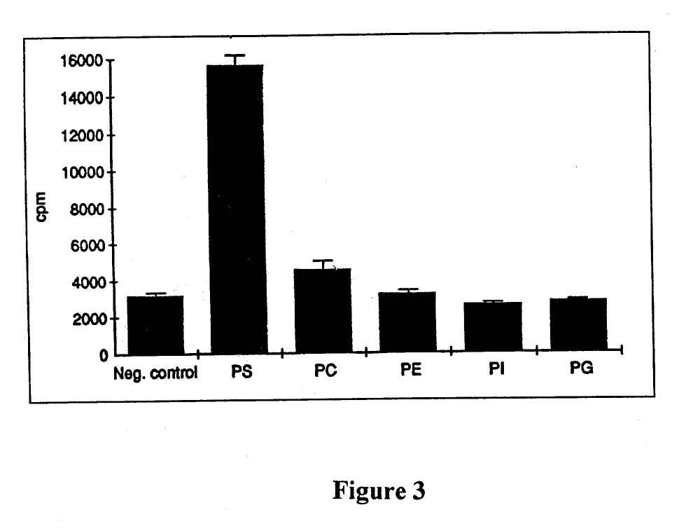

The cDNA encoding human Syn-1 (p65) was then used to express the p45 form of Syn-1 in the pGEX-KG prokaryotic expression system (Pharmacia) as a GST fusion protein. Recombinant human p45 Syn-1 was expressed in the pET3 expression system, purified by heparin affinity and RP-HPLC HPLC, iodinated using lactoperoxidase methods according to Engleka et al. (1992), and added to plastic wells coated with 10 μM phospholipids as described by Perin et al., Nature 345:260-263 (1990). Negative control, no phospholipid; pS, phosphatidylserine; pC, pl choline; pE, pL ethanol-amine; pI, pL inositol; and pG, pL glycerol. Data reported as CPMs bound (± std deviation) after three washes with 0.1M Tris, pH 7.4 containing 0.15 M NaCl. As shown in FIG. 3, the pLS-binding property of p45 Syn-1 was confirmed.

Iodination of the recombinant p45 protein permitted the characterization of p45 Syn-1 as a heparin-binding protein which elutes from immobilized heparin at 0.6M NaCl (data not shown). Interestingly, the p45 protein, originally characterized in 1985 as a heparin-binding protein, eluted with FGF-1 at 1.0M NaCl. The apparent discrepancy in the property of heparin elution was explained as an interaction between p45 Syn-1 and FGF-1, or as a post-translational modification of p45 Syn-1.

### Example 2

### FGF-1 IS Present in Temperature-conditioned Medium as a High Mᵣ Complex

Although evidence points to interaction between p45 Syn-1 and FGF-1, to date conventional methods (gel analysis, exclusion chromatography and ligand affinity chromatography at a variety of pH and ionic strengths) have failed to provide evidence of direct interaction between these recombinant proteins. Consequently, experiments were designed to determine whether SDS was interfering with the resolution of FGF-1 as a large Mᵣ complex.

Immunoblot analysis of conditioned medium from heat shocked FGF-1 NIH 3T3 transfectants was activated by addition of either DTT or (NH₄)₂SO4, absorbed to heparin Sepharose, and gradient eluted with NaCl. A non-reduced, limited SDS-Page FGF-1 immunoblot was performed using FGF-1 antiserum at pH 7.2 on the samples as described by Jackson et al. (1992). SDS was eliminated from the running gel.

As shown in FIG. 4, FGF-1 can be clearly identified as a single band with a characteristic heparin elution profile. However, although FGF-1 eluting from immobilized heparin near 1.0M NaCl was present as a single band, FGF-1 eluting from the heparin column prior to 0.6M NaCl were resolved as multiple bands. Because proteins may migrate in the limited SDS-PAGE gel on the basis of both size and charge, it is not possible to assign accurate Mᵣ to these bands. Hence Mᵣs have been assigned for comparative purposes. However, the results have been interpreted as indicating that high Mᵣ FGF-1-containing complexes can be identified by limited SDS-PAGE analysis of (NH_{4 2}) SO₄-activated, but not from DTT-activated heat-shocked conditioned medium from NIH 3T3 FGF-1 transfectants.

The sensitivity of the high Mᵣ FGF-1 containing complexes also demonstrates the significance of FGF-1 cysteine residues in complex formation. Purification of the FGF-1 present in the 0.2-0.4M NaCl-eluted fractions yielded FGF-1 as a homodimer.

### Example 3

### FGF-1 is a Phosphatidylserine (pS)-Binding Protein

An examination of the ability of FGF-1 to associate with phospholipids using conventional phospholipid-binding methods suggested that while FGF-1 is able to associate with pS, FGF-1 does not recognize other phospholipids (Lippincott-Schwartz et al. (1989)). It was possible to utilize synthetic peptides to map the domain within FGF-1 responsible for ps-binding, and the domain defined by residues 112-138, which has been previously characterized as the dominant heparin-binding domain in FGF-1 (Burgess et al., J. Cell Biol. 111:2129-2138 (1990)), and which appeared to be involved in the binding to pS (Tarantini et al., (1995)). In addition, the cys-free FGF-1 mutant which binds heparin and is active as a BALB/c 3T3 cell mitogen was not able to associate with pS. Id.

To confirm the FGF-1 ps-binding activity established by these solid phase binding assays, novel gel retardation methods were used to visualize the FGF-1:pS complex. Native PAGE (pH 6.8) analysis was performed with varying amounts of phosphatidylserine (pS) and phosphatidylethanolamine (pEA), but with a constant amount of [¹²⁵I]-FGF-1. The pL were premixed with cold FGF-1 (10 ng), followed by the addition of the [¹²⁵I]-FGF-1 probe, and the entire sample was subjected to electrophoresis with the exception of the last lane in each gel which contained 50 μg of either pS or pEA containing cold FGF-1 (10 ng) which was loaded without the probe. In this situation, [¹²⁵I]-FGF-1 was added to the top of the lane immediately prior to electrophoresis in an attempt to visualize the level of non-specific trapping of the probe in the pL micelle.

As shown in FIG. 5, the migration of FGF-1 in native PAGE gels can be significantly retarded by the presence of increasing concentrations of pS. In contrast, pEA was inefficient in retarding the electrophoretic mobility of FGF-1. The combined data indicates that FGF-1 is a pS-binding protein, suggesting that the interaction between Syn-1 and FGF-1 involves an interaction between the phospholipid component of these proteins as a co-factor, and that the interaction is involved in the regulation of FGF-1 export Indeed, ligand affinity chromatography with immobilized FGF-1 and/or Syn-1 and conventional gel exclusion chromatography is used in the presence and absence of pS to monitor the formation of an FGF-1:Syn-1 complex as a means of re-evaluating the earlier results.

### Example 4

### The Role of CU²⁺ Oxidaton to Induce Heterodimer Formation Between FGF-1 and P45 SYN-1

In view of the following previously established principles: (i) reducing agents are able to activate the latent form of FGF-1 resolved in temperature-conditioned medium as an inefficient heparin-binding complex, (ii) both Syn-1 and FGF-1 are heparin- and pLS-binding proteins, (iii) Cu²⁺ is able to induce FGF-1 homodimer formation, and (iv) FGF-1 cys residues are critical for FGF-1 export, experiments were designed to examine the ability of Cu²⁺ to oxidized Syn-1 homodimer formation and Syn-1:FGF-1 heterodimer formation.

Purified FGF-1 and p45 Syn-1 were incubated with, and without, Cu²⁺ for 10 min. and resolved on two non-reduced SDS-PAGE gels (A and B), as described by Maciag et al., Recent Prog. Horm. Res. 49:105-123 (1993). The systems were identical except gel SB was immunoblotted with FGF-1 antibody, whereas gel SA was not.

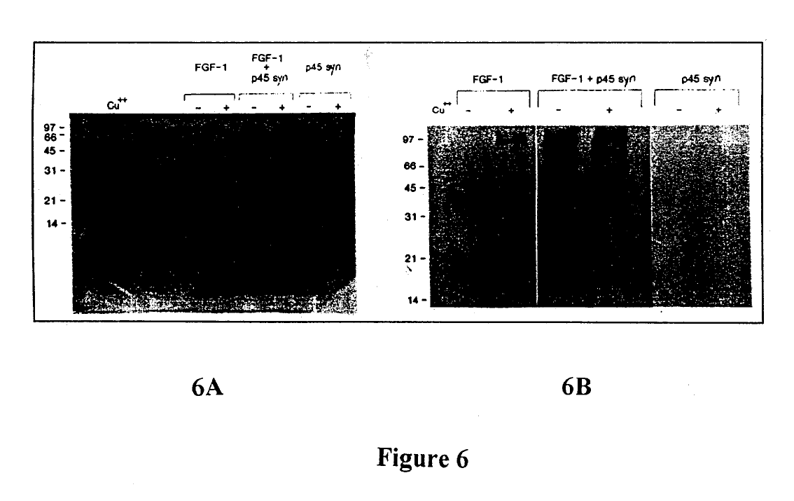

As shown in FIG. 6A, Cu²⁺ is able to induce the formation of the FGF-1 homodimer but was not able to induce the formation of a Syn-1 homodimer. However, when FGF-1 and p45 Syn-1 were added together, non-reduced SDS-PAGE was able to resolve a unique component with an apparent Mᵣ ˜66,000 (FIG. 6A). To determine whether this band contained FGF-1, this experiment was repeated and analyzed by FGF-1 immunoblot analysis. As shown in FIG. 6B, the band previously resolved as a putative Syn-1:FGF-1 heterodimer contained FGF-1.

### Example 5

### C-Localization of Syn-1 and FGF-1 as a High M_{R} Cowlex in temperature Conditioned Medium

In view of the following previously established principles: (I) FGF-1 and p45 Syn-1 are both pS-binding proteins, (ii) FGF-1 and Syn-1 may associate with each other through a pS:protein rather than in a direct protein:protein interaction, and (iii) FGF-1 can be resolved as a low ionic strength heparin-binding and DTT sensitive high Mᵣ complex in temperature-conditioned medium activated with (NH₄)₂S0₄ (Jackson et al. (1995); Tarantini et al. (1995)), experiments were designed utilizing the limited SDS-PAGE system in which SDS is removed from the sample buffer to evaluate the co-localization of FGF-1 and Syn-1. The reasoning was that if sufficient SDS was present in the sample buffer to electrophoretically resolve the sample as a function of Mr, it would be insufficient to disrupt any potential pL:protein interaction.

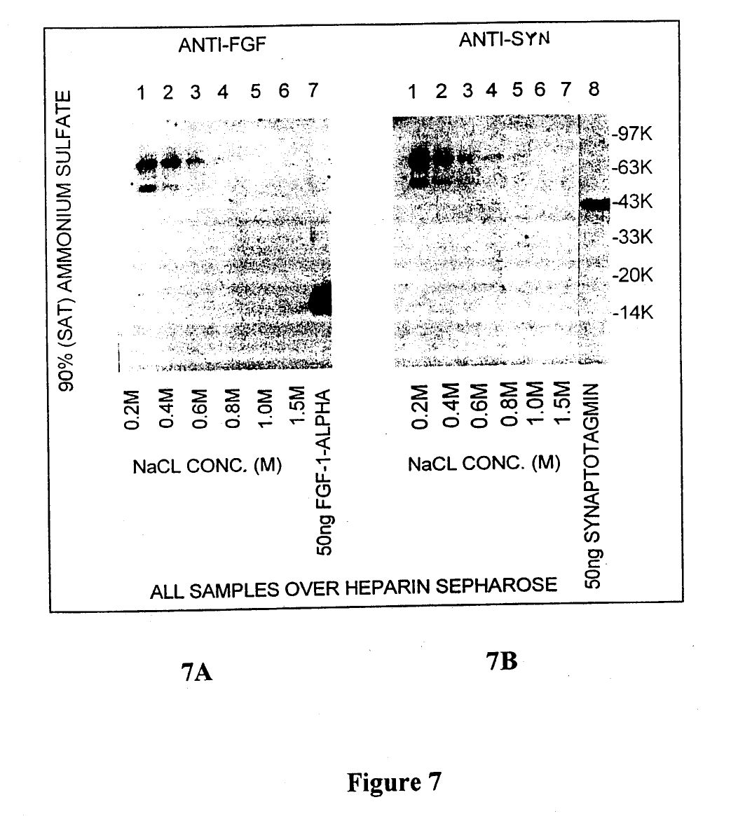

Following the protocol described in Example 2, an FGF-1 immunoblot analysis was conducted using the non-reduced, limited SDS-PAGE system to resolve (NH₄)₂S0₄-activated heat shock conditioned medium from NIH 3T3 cell FGF-1 transfectants, previously absorbed to heparin-Sepharose and gradient eluted with NaCl. As shown in FIG. 7B, FGF-1 was identified as a high Mᵣ complex in the low NaCl elution fractions (<0.6M) and as a low Mᵣ protein in the high NaCl elution fractions (>1.0M).

After this immunoblot was stripped, the same blot was probed with a Syn-1 antibody. As shown in FIG. 7B, the low Mᵣ FGF-1 signal (Lane 6) and the recombinant FGF-1 standard (Lane 7) were not detected; however, the high Mᵣ signals previously detected with an FGF-1 antibody were also detected by the Syn-1 antibody only in those samples which were eluted from immobilized heparin as low ionic strength heparin-binding proteins (Lanes 1,2,3). The combined data indicate that both FGF-1 and Syn-1 can be co-localized using the non-reduced, limited SDS-PAGE system as high Mr complexes eluting from heparin-Sepharose with a NaCl gradient profile similar to that described in FIG. 4 for the FGF-1 homodimer. Indeed, these data support the initial premise that FGF-1 is able to interact with Syn-1 in heat shock conditioned medium. Further, purification of FGF-1 from the 0.2-0.4M NaCl-eluted fractions has been shown to resolve FGF-1 in these fractions as a homodimer (Jackson et al. (1995)).

### Example 6

### Stable Antisense (γ)-Syn-1, Sense FGF-1 NIH 3T3 Cell Co-Transfectants Exhibit Poor Growth, an FGF-1 Growth Response and Do Not Export FGF-1 in Response to Heat Shock

To further evaluate the role of Syn-1 in the heat shock-induced FGF-1 export pathway, the NIH 3T3 cell FGF-1 transfectants were co-transfected with an antisense (γ)Syn-1 cDNA (1410 bp) designed to repress the translation of the endogenous Syn-1 mRNA using a hygromycin-resistant expression plasmid. The reasoning behind this experiment was that if Syn-1 was involved in the regulation of the FGF-1 export pathway, it should be possible to at least attenuate the release of FGF-1 in response to temperature stress under conditions where the translation of the Syn-1 mRNA is repressed. A Syn-1 cDNA fragment encoding the Syn-1 open-reading frame was used for these studies rather than the more traditional antisense-AUG oligomer approach because the latter method is more appropriate for the translational repression of low abundance transcripts; plus stable clones could be obtained for further biochemical analysis.

The experimental design and protocols were identical to those described in Jackson et al. (1995), and Tarantini et al. (1995). Two stable clones were evaluated including one in which the intracellular levels of FGF-1 were reduced as a result of the selection process. The pXZ38 cells were used as control transfectants, and #10 Syn-1 and #19 Syn-1 are the low and high FGF-1 expressing γ-Syn-1, FGF-1 NIH 3T3 cell co-transfectants, respectively.

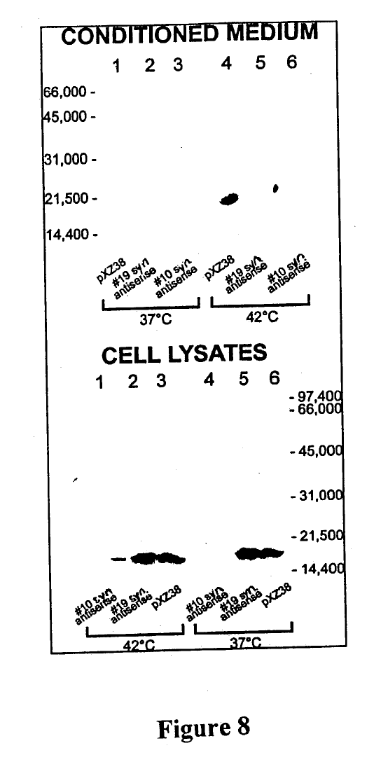

As shown in FIG. 8, FGF-1 was not observed in medium conditioned by heat shock in either of the co-transfectant clones, suggesting that the repression of Syn-1 translation inhibits the release of FGF-1 in response to heat shock. This further supports the conclusion that Syn-1 is involved in the regulation of FGF-1 export.

FIG. 8 shows that antisense (γ)Syn-1 represses the release of FGF-1 in response to heat shock. Immunoblot analysis of conditioned medium from heat shocked FGF-1 NIH 3T3 transfectants cotransfected with antisense (γ)Syn-1 cDNA, as compared with control conditioned media; and cell lysates under comparable conditions. pXZ38 cells=control transfectants; #10 Syn-1 and #19 Syn-1 are low and high FGF-1 expressing γ-Syn-1, FGF-1 NIH 3T3 cell co-transfectants, respectively. The experimental design and procedures used are as described in Jackson, et al., J. Biol. Chem. 270: 33-36 (1995) and Tarantini, et al., J. Biol. Chem. (1995)).

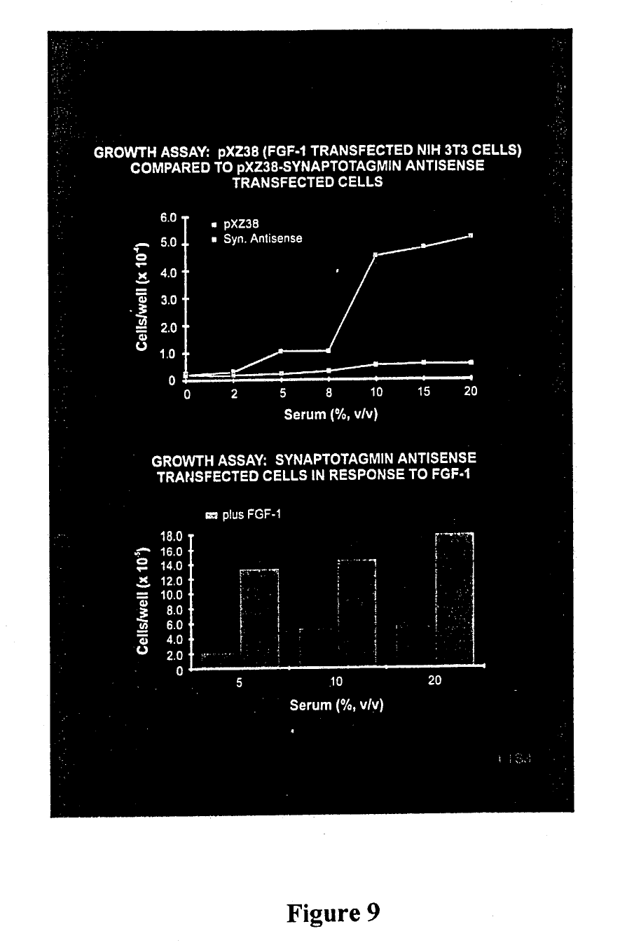

As aside, it was noted that the γ-Syn-1, sense FGF-1 co-transfectants grew slowly in response to serum (FIG. 9). A fetal bovine serum (FBS) growth response curve shows FGF-1 and γ-Syn-1, FGF-1 co-transfectants as a function of FBS (FIG. 9A); and a second curve shows the growth response of γ-Syn-1, FGF-1 co-transfectants (FIG. 9B) as a function of FBS in the presence and absence of FGF-1 (10 ng/ml containing 5 μg/ml heparin). The cells were seeded at a density of 1×10⁴ cells/cm², and a viable cell number was reported after 5 days.

However, perhaps even more interesting, it was seen in FIG. 9 that the addition of exogenous FGF-1 to these cells significantly increased their ability to grow in the presence of 20% FBS. When tested, stable γ-Syn-1 NIH 3T3 cell transfectants (lacking the FGF-1 cDNA) exhibited similar growth properties. This seems to be the first demonstration that NIH 3T3 cells can exhibit FGF-1-dependent growth in the presence of 20% FBS, an observation very reminiscent of the well defined growth properties exhibited by human endothelial cells.

### Example 7

### The FGF-1 Export Pathway does not Restrict the Release of a Large Molecular Weight (M_{R}) Form of FGF-1

In order to determine whether a size restriction exists preventing high Mᵣ complexes from exiting the cell, a chimera was prepared in which the reporter gene, β-galactosidase (gal), was ligated to the carboxy-terminus of FGF-1 (COOH-terminal β-gal chimera) as previously described (Zhan et al., Biochem. Biophys. Res. Commun. 188:982-991 (1992)). In addition, another chimera was also constructed in which the nuclear localization signal (NLS) from the SV40 large T antigen (SV40T) gene was ligated at the amino-terminus of FGF-1:β-gal (NH₂-terminal SV40T NLS COOH-terminal β-gal chimera). These constructs were transfected into NIH 3T3 cells and stable transfectants obtained and examined for the cytosolic and nuclear presence of the reporter gene using either immunofluorescence microscopy with an anti-β-gal antibody or X-gal staining as described by Zhan et al. (1992).

As shown in FIG. 10A, X-gal and immunofluorescence staining for the β-gal reporter gene demonstrates cytosolic staining for the FGF-1:β-gal chimera, but nuclear staining for the SV40T-NLS:FGF-1:β-gal protein. In FIG. 10A, the reporter gene may be seen in the nucleus of the SV40T NLS:FGF-1:β-gal transfectants and in the cytosol of the FGF-1:β-gal transfectants.

The stable transfectants were also subjected to heat shock (2 hrs, 42° C.), and the conditioned medium processed for β-gal immunoblot analysis as described by Jackson et al. (1995), and Tarantini et al., J. Biol. Chem. (1995) (see, Example 6), and immunoblot analysis for β-gal performed. The cells were lysed in boiling SDS which does not conserve the nuclear compartment and the total cell lysate processed as described by Zhan et al. (1992).

As shown in FIG. 10B, while FGF-1:β-gal was readily detected in all NIH 3T3 cell lysates and in the heat shock conditioned medium, the SV40T-NLS:FGF-1:β-gal protein was only present in the NIH 3T3 cell lysates. The fact that the SV40T-NLS:FGF-1:β-gal protein was not detected in the heat shock conditioned medium, indicates that intranuclear FGF-1 is probably not accessible to the components involved in regulating the FGF-1 export pathway. Thus, placement of intracellular FGF1 in an organelle which restricts its access to Syn-1, completely attenuates the temperature-induced FGF-1 export pathway. However, the presence of FGF-1:β-gal in heat shock conditioned medium as a Mᵣ˜118 kDa chimera argues that the mechanism used to release FGF-1 does not exclude high Mᵣ forms of FGF-1. Thus, high Mᵣ FGF-1:Syn-1-contaning complexes are probably restricted from the FGF-1 export pathway.

In FIG. 10B, the FGF-1:β-gal and SV40T NLS:FGF-1 β-gal both appear to migrate with an apparent Mᵣ 118 kDa since the SV40T NLS only contributes 6 amino acid residues to the total mass of the chimera.

### Example 8

### The Identification of Putative FGF-1-Associated Proteins Present in the Temperature-Conditioned Medium OF FGF-1:β-Gal NIH 3T3 Cell Transfectants

Because the presence of the FGF-1:β-gal chimera could not be resolved in medium conditioned by heat shock (FIG. 10B) as a structurally intact protein, experiments were designed to determine whether immunoprecipitation strategies could be utilized to resolve the presence of proteins associated with FGF-1 using (³⁵S)-met/cys labeled transfectants. There was concern about using either Syn-1 or FGF-1 antibodies for this purpose because similar strategies employed in receptor-mediated signaling, DNA and mRNA transacting factors, and intracellular trafficking arts have suggested that antibodies against the protein of interest that are used to immunoprecipitate the protein of interest as a non-covalent multi-protein complex can disrupt the conformational integrity of the complex, and thus irreversibly modify associative protein:protein interactions. Consequently, the FGF-1:β-gal chimera secretion data produced by these experiments are particularly significant because they indicate the possibility of using β-gal antibodies for this purpose in a non-disruptive manner since β-gal-transfected NIH 3T3 cells do not release β-gal into the conditioned medium in response to heat shock (data not shown).

Moreover, immunoprecipitation of (³⁵S)-met/cys-labeled β-gal NIH 3T3 cell transfectants resolved only the presence of β-gal as a radiolabeled protein from cell lysates prepared by Dounce homogenization (data not shown). Thus, it was determined that if β-gal immunoprecipitation from either cell lysates or medium conditioned by heat shock of FGF-1:β-gal NIH 3T3 cell transfectants were able to resolve multiple radiolabeled proteins, the bands would presumably represent proteins associated with FGF-1, and not β-gal. Of course, one cannot eliminate the possibility that the protein resolved by β-gal immunoprecipitation are associated with both FGF-1 and β-gal.

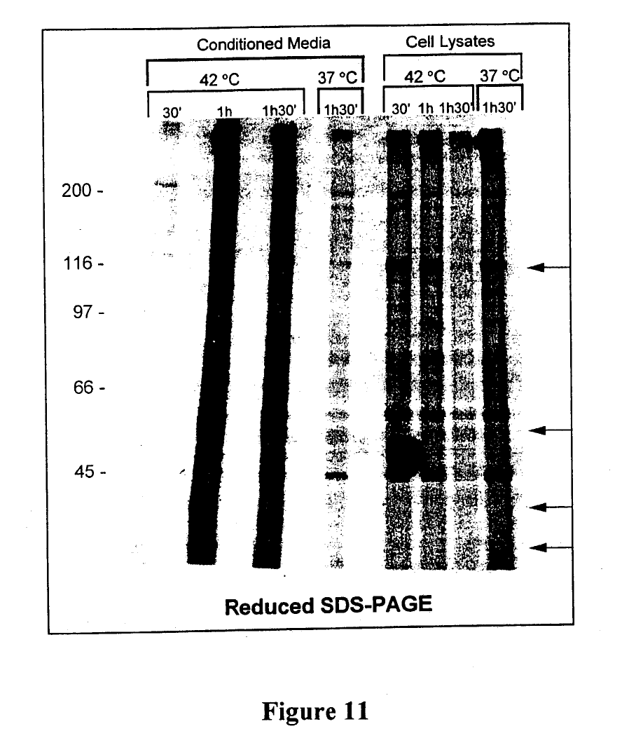

Thus, FGF-1:β-gal NIH 3T3 cell transfectants were metabolically labeled with (³⁵S)-met/cys and subjected to heat shock (42° C., 2 hr). Cell lysate and conditioned medium were immunoprecipitated with anti-β-gal antiserum and analyzed by SDS-PAGE. As shown in FIG. 11, the immunoprecipitate resolved not only the presence of the FGF-1:β-gal chimera (Mᵣ˜118 kDa) in the medium conditioned by heat shock, and not in control conditioned medium; it also resolved additional polypeptides with apparent Mᵣs of approximately 70, 40 and 20 kDa (marked with arrows in FIG. 11). Similar radiolabeled bands were identified in the heat shock cell lysate but not in the control lysate. The combined data suggest that these bands probably represent proteins associated with each other as a high Mᵣ complex; and since the FGF-1:P-gal chimera is the target of this complex, they are presumed to be FGF-1:β-gal-associated proteins.

### Example 9

### The Identification of Other Proteins Associated with the FGF-1:Syn-1 Complex

After the inventors determined that a p40 fragment of Syn-1 is complexed with the FGF-1 homodimer in medium from heat shocked FGF-1 NIH 3T3 cells transfected in vitro, and in view of the knowledge that FGF-1 derived from bovine tissue extracts exhibits the presence of FGF-1 as a high molecular weight, non-covalent complex, the FGF-1:Syn 1 complex was further examined to determine whether or not other proteins were associated with the complex. Immunoblot analysis of fractions resolved by heparin affinity chromatography identified a fraction of ovine brain extract in which both FGF-1 and Syn-1 were present. This fraction, when further resolved by RP-HPLC, revealed multiple fractions containing FGF-1, Syn-1, as well as a fraction containing both FGF-1 and Syn-1. See, FIG. 14, top panel.

Since the fraction containing both FGF-1 and Syn-1 potentially represented a complex of these two proteins, an assay was undertaken to separate the FGF-1:Syn-1 fraction from the other containment proteins, if any. Thus the fraction containing both FGF-1 and Syn-1 was collected, and further resolved by RP-HPLC. As shown in the middle panel of FIG. 14, the fraction containing both FGF-1 and Syn-1 eluted with a retention time similar to that seen in the previous observation (FIG. 14, top panel), thus confirming the presence of the FGF-1 and Syn-1 proteins in this fraction.

Since the fraction represented a potentially stable complex between FGF-1 and Syn-1, a study was designed to determine whether FGF-1 and Syn-1 could be dissociated using a denaturant. Consequently, the fraction was treated with 8M guanidinium HCl, a chaotropic agent, and the fraction was again resolved by RP-HPLC. As shown in the bottom panel of FIG. 14, multiple peaks were generated from the putative FGF-1:Syn-1 complex, including peaks with retention times similar to that which had previously been identified as FGF-1 and Syn-1. In addition, the FGF-1 and Syn-1 analysis confirmed the identity of these peaks.

Surprisingly, however, additional peaks with different elution retention times were also generated by treatment of the FGF-1:Syn-1 complex with a chaotropic agent. This demonstrated that FGF-1 is not only complexed with Syn-1 in neutral pH extracts of ovine brain, but also with other proteins. Further, it was determined that this complex is remarkably stable to exposure to RP-HPLC solvents.

Conventional protein sequencing methods, however, failed to identify the other proteins associated with either FGF-1, Syn-1 or both in the Syn-1:FGF-1 complex (FIG. 14, bottom panel). Moreover, Edman degradation of the individual peaks resolved by RP-HPLC did not yield an NH2-terminal sequence, showing that each of the components of the FGF-1:Syn-1 complex contain a blocked NH₂-terminus, including FGF-1 and Syn-1. This determination suggested that the purification of the multiprotein FGF-1:Syn-1 complex can be achieved under conditions which do not result in the hydrolytic modificaton of the NH₂-terminus of any component of this complex.

Since the identity of the individual protein components of the FGF-1:Syn-1 complex could not be accessed by direct NH₂-terminal sequence analysis, peptide maps of those peaks exhibiting prominent absorbance profiles were obtained Analysis of the most prominent peaks by NH₂-terminal sequence analysis of multiple proteinase K-derived peptides identified the first peak to be identical to the ovine homolog of human S100A13. Similar analysis of the second prominent absorbance peak demonstrated it to be identical to the ovine homolog of human cyclophilin B. Thus, the present data clearly identifies S100A13 and cyclophilin B to be components of the FGF-1:Syn-1 complex.

S100A13 is a recently identified member of the human S100A gene family with well conserved structural features. Characterization of the isolated S100A13 gene has shown that it contains the prerequisite structural motifs identifying the S100A gene family members as Ca²⁺-, calmodulin-, and acidic phospholipid-binding proteins. In addition, S100A13 is a cysteine-free structure which is exported even though, like FGF-1, it lacks a classical signal peptide sequence to direct it through the conventional ER-Golgi apparatus-mediated exocytotic pathway. Nevertheless, S100A proteins are well characterized as a calmodulin-binding protein, and are involved in the regulation of the filamentous cytoskeleton. Data suggests that FGF-1 may utilize the cytosolic side of conventional Syn-1 containing vesicles for traffick to the inner surface of the plasma membrane. Thus, S100A13 may serve as a conduit between the exocytotic organelle and the calmodulin-rich F-actin cytoskeleton. In addition, data suggest that (i) the biologic activities previously associated with the S100A gene family members may actually be the result of an FGF gene family contaminant found in relatively low specific activity preparations, and (ii) S100A antagonists could prove useful to limit the release of FGF-1.

The other component of the multiprotein complex, cyclophilin B (CpB) is a cyclosporin A1 (CSA)-binding protein, which was initially identified as a heat shock-induced peptidylproline CIS-trans isomerase (hsp4o) involved in protein-folding and protein-protein interactions. CsA is well recognized as an immune suppressor in humans, having novel tissue/organ-specific side effects, including enhanced peripheral nerve repair in man.

Thus, the data provide evidence to support an association of Syn-1 and FGF-1 as a multiprotein complex in neural tissue in vivo, and to confirm the characterization of the FGF-1:Syn-1 complex derived from in vitro systems. In addition, because two additional proteins present within the FGF-1:Syn-1 complex have been identified as S100A13 and cyclophilin B, it is assumed that agents which agonize or antagonize this association will provide useful reagents for the inhibition and/or activation of the FGF-1 secretion pathway in vitro and in vivo.

### Example 10

### Isolating Syn-1-CE

Because the data in FIG. 7 indicated that Syn-1 and FGF-1 are present in heat-shocked condition medium as a high Mᵣ complex which elutes from heparin-Sepharose at 0.4M NaCl, this system was tested. A neutral extract of bovine brain was prepared as described by Jaye et al. (1986), and resolved by 0.4M NaCl elution from heparin-Sepharose. The 0.4M NaCl eluted fractions were pooled, lyophilized, and the fraction resolved by Sephadex G-100 gel exclusion chromatography at pH 7.0 as previously described by Maciag et al. (1982). The fractions were pooled and subjected to either immunoprecipitation with a Syn-1 antibody followed by immunoblot analysis using an FGF-1 antibody (data not shown) or immunoprecipitation with an FGF-1 antibody followed by reduced immunoblot analysis using a Syn-1 antibody (FIG. 13).

The samples from the Sephadex G-100 column were Western blotted as described by Zhan et al., J. Biol. Chem. 269:20221-20224 (1994). The gel on the left of FIG. 13 represents FGF-1 immunoprecipitation followed by Syn-1 immunoblot analysis. The gel on the right of FIG. 13 represents Syn-1 immunoprecipitation followed by Syn-1 immunoblot analysis.

If the high Mᵣ fractions contained FGF-1 homodimer:Syn-1 complex, it is possible to not only visualize FGF-1 in the Syn-1 immunoprecipitate, but also to visualize a Syn-1 fragment in the FGF-1 immunoprecipitate. Indeed, it was possible to detect FGF-1 in the Syn-1 immunoprecipitate and the p45 Syn-1 fragment in the FGF-1 immunoprecipitate in the high M, Sepharose G-100 fractions (FIG. 13). The combined data not only confirm the data derived from the cell culture effort relative to the association between a Syn-1 fragment and FGF-1 using a physiologic relevant organ, but they also predict that the neutral pH brain extract may contain a protease or proteolytic-type of a complex, which was able to successfully modify p65 Syn-1 and convert it to p45 Syn-1.

The Syn-1-CE open-reading frame is defined and characterized as previously described for other novel cDNAs (see, e.g. Zhan et al. (1993); Hla et al, Biochem. Biophys. Res. Comm. 167:637643 (1990); Hla et al., J. Biol. Chem. 265:9308-9313 (1990)), including the identification of putative structure-function domains, motifs and/or sites. This provides an appropriate resource for the expression of Syn-1-CE as a recombinant protein using the pET3c prokaryotic expression plasmid either as a non-modified protein or as a GST fusion protein as described (Zhan et al (1994)).

The Syn-1-CE recombinant is purified using either the GST tag or by conventional protease affinity methods, permitting this resource to be used not only for the generation of Syn-1-CE antibodies, but also as an (¹²⁵I)-ligand to probe to determine whether Syn-1 and/or FGF-1 homodimer/monomer associate with Syn-1-CE using reduced/non-reduced immunoprecipitation/ immunoblot analysis as described in FIG. 13. The Syn-1-CE antibody will also prove useful to study the biosynthesis of Syn-1-CE using conventional pulse-chase in either ECV and/or SMC FGF-1₁₋₁₅₄ transfectants relative to the synthesis of Syn-1.

Although the present invention has been described with reference to the presently preferred embodiments, the skilled artisan will appreciate that various modifications, substitutions, omissions and changes may be made without departing from the spirit of the invention. Accordingly it is intended that the scope of the present invention be limited only by the scope of the following claims, including equivalents thereof.

We claim:

1. A method of regulating FGF-1 export from a cell comprising administering a therapeutic agent in an amount sufficient to inhibit the export of FGF-1, wherein said therapeutic agent inhibits the formation of a complex comprising FGF-1, and wherein the formation of said complex is a prerequisite to FGF-1 export.

2. A method according to claim 1, wherein said complex further comprises Syn-1.

3. A method according to claim 1, wherein said therapeutic agent inhibits the release of FGF-1 from a complex comprising FGF-1 and Syn-1.

4. A method according to claim 1, wherein said therapeutic agent inhibits the release of FGF-1 from, or the association of FGF-1 with, an intracellular vesicle.

5. A method of regulating FGF-1 export from a cell comprising administering a therapeutic agent in an amount sufficient to promote the export of FGF-1, wherein said therapeutic agent promotes the formation of a complex comprising FGF-1, and wherein the formation of said complex is a prerequisite for FGF-1 export.

6. A method according to claim 5, wherein said complex further comprises Syn-1.

7. A method according to claim 5, wherein said therapeutic agent promotes the release of FGF-1 from a Syn-1 at or near the plasma membrane.

8. A method according to claim 5, wherein said therapeutic agent promotes the release of a complex comprising FGF-1 from an intracellular vesicle.

9. A method according to claim 5, wherein said therapeutic agent promotes the association of a complex comprising FGF-1 with an intracellular vesicle.

10. A method of regulating FGF-1 export, comprising inhibiting a protease that cleaves Syn-1 in an FGF-1:Syn-1 complex, thereby inhibiting the export of said FGF-1:Syn-1 complex.

11. A method of regulating FGF-1 export, comprising increasing the activity of a protease that cleaves Syn-1 in an FGF-1:Syn-1 complex, thereby promoting the release of FGF-1 at or near the plasma membrane.

12. A method according to claim 1, wherein said therapeutic agent is selected from the group consisting of:
a. an antibody;
b. an immunologically active fragment of an antibody; and
c. an anti-inflammatory agent.

13. A therapeutic agent which regulates the export of FGF-1 from a cell, wherein said agent:
a. inhibits export of FGF-1;
b. does not inhibit secretion of a leader sequence-containing protein; and
c. inhibits the binding between the FGF-1 and an intracellular vesicle.

14. A therapeutic agent according to claim 13, wherein said agent inhibits the formation of an FGF-1:Syn-1 complex.

15. A therapeutic agent according to claim 13, wherein said agent inhibits the release of FGF-1 from a complex comprising FGF-1.

16. A therapeutic agent according to claim 13, wherein said agent inhibits the association of FGF-1 with an intracellular vesicle.

17. A therapeutic agent according to claim 13, wherein said agent inhibits the release of FGF-1 from an intracellular vesicle.

18. A therapeutic agent according to claim 13, wherein said agent is selected from the group consisting of:
a. an antibody;
b. an immunologically active fragment of an antibody; and
c. an anti-inflammatory agent.

19. A therapeutic agent according to claim 18, wherein said agent is a protein derived from a recombinant molecule.

20. An isolated, purified protease that cleaves Syn-1 in an FGF-1:Syn-1 complex and which thereby regulates the export of FGF-1.

21. An FGF-1 homodimer:Syn-1 complex.
